(19) United States
(12) Patent Application Publication
(10) Pub. No.: US 2009/0272806 A1
(43) Pub. Date: Nov. 5, 2009

(54) Instrument tracking

(75) Inventors: Todd Mitchell Kemp, Coorparoo (AU); Jennifer Frances Kemp, Coorparoo (AU)

Correspondence Address: EDWARDS ANGELL PALMER & DODGE LLP, P.O. BOX 55874, BOSTON, MA, 02205, US

(73) Assignee: Romeena Pty Limited as Trustee for Kemp Family Trust, Coorparoo (AU)

(21) Appl. No.: 11/920,953

(22) PCT Filed: May 25, 2006
(86) PCT No.: PCT/AU2006/000700

§ 371 Date: Jul. 1, 2009

Related U.S. Application Data
(60) Provisional application No. 60/689,663, filed on Jun. 9, 2005.

(30) Foreign Application Priority Data
May 25, 2005 (AU) 2005902682

(51) Int. Cl. G06K 9/80; G06K 9/62; G06K 19/06

(52) U.S. Cl. 235/462.1; 235/470; 235/492

(57) ABSTRACT

A method of tracking a medical instrument. The method includes using a processing system to receive indicating data from a scanning device, the indicating data being indicative of a unique identifier and being determined when the scanning device is used to scan the instrument. The processing system uses the identifier to obtain instrument data associated with the instrument from a database, before interacting with the instrument data to either record one or more events performed with the instrument or determine one or more events performed with the instrument.

### BACKGROUND OF THE INVENTION

The present invention relates to a method and apparatus for tracking instruments, and in particular to tracking the use of medical instruments in a medical environment. The present invention also relates to a method and apparatus for tracking assets within a medical environment, as well as a method and apparatus for ordering stock.

### DESCRIPTION OF THE PRIOR ART

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

There is a need within the medical industry to be able to track medical instruments throughout their life to thereby monitor the procedures in which the medical instruments have been used, together with details of their cleaning operations and the like. This is important for situations in which the instruments have either been used in a procedure where there is a risk of instrument contamination, or when problems are discovered with the sterilisation process, and there is a consequent risk of contamination or infection of patients. In these situations it is useful to be able to identify those patients on which the instruments have been used, thereby allowing the patients to be examined to check there has not been any contamination or infection.

However, it is difficult to reliably mark medical instruments. Existing techniques generally use laser engraving or the like. However, medical instruments are typically formed from a range of different alloys, depending on factors such as the quality and source of the instrument, and each of the alloys will have different properties. As laser engraving is dependent upon the properties of the material being engraved, this means that any laser used in such a progress must be configured and subsequently calibrated for each instrument to be marked. Thus, for example, instruments made with different materials would require different laser configurations for successful engraving to be achieved. This results in significant expenditure when arranging to encode a number of instruments.

In addition to this, the engrave process creates surface relief structures which can in turn cause difficulties in suitable cleaning of the instrument, as well as effecting the strength of the instrument in some cases.

Accordingly, instrument tracking is generally performed on a per tray basis. In this case, when medical instruments are used in procedures the medical instruments are provided as part of a tray. The use of the tray can be monitored allowing instruments to be tracked. However this suffers from a number of disadvantages.

In particular, as instruments cannot be distinguished, this requires that instruments are always used with the same tray. As a result, if any one instrument is unavailable for use, for example due to the problems with sterilisation in the cleaning process, the entire tray cannot be used until the instrument is replaced. This results in significant problems in tray supply. Secondly, it is possible for instruments on different trays to be mixed up, which in turn leads to inaccuracies in the tracking procedure.

In addition to this, irrespective of the marking procedures used, it is often difficult to track the use of assets, such as medical instruments, and other items such as consumables, within a hospital. In this instance, this is important not only from the point of view of ensuring that sufficient stock is maintained, but also to make sure that it is possible to subsequently identify what assets have been used on a patient, or during procedures undergone by a patient. This can be important both from the perspective of inventory management, and subsequently identifying used assets in the case in which an investigation is required.

It is therefore desired to ameliorate one or more of the above mentioned problems.

### SUMMARY OF THE PRESENT INVENTION

In a first broad form the present invention provides a method of tracking a medical instrument, the method including, in a processing system:
a) receiving indicating data from a scanning device, the indicating data being indicative of a unique identifier and being determined when the scanning device is used to scan the instrument;
b) obtaining, from a database and using the indicating data, instrument data associated with the instrument; and,
c) interacting with the instrument data to perform at least one of:
   i) recording one or more events performed with the instrument; and,
   ii) determining one or more events performed with the instrument.

Typically the medical instrument has thereon or therein a machine readable code encoding the unique identifier, and wherein the indicating data is determined when the scanning device is used to scan the code.

Typically the unique code is determined by:
a) imaging the medical instrument using an infra-red scanner to thereby generate an image; and,
b) generating the unique identifier by applying a predetermined algorithm to the image.

Typically the method further includes, in the processing system:
a) displaying at least some of the instrument data; and,
b) receiving, via an input device, input commands confirming that the instrument has been correctly identified; and,
c) interacting with the instrument data following a successful confirmation.

Typically the event includes cleaning the instrument as part of a batch of instruments, and wherein the method includes, in the processing system:
a) generating batch data indicative of at least one of:
   i) a time of cleaning;
   ii) a date of cleaning;
   iii) a cleaning process type;
   iv) cleaning process properties; and,
   v) an indication of one or more individuals performing the cleaning; and,
b) recording an association between the batch data and the instrument data of each instrument in the batch.

Typically the method includes, in the processing system, and following cleaning of the instrument updating list data representing instruments available for use in a procedure.

Typically the event includes performing a medical procedure, and wherein the method includes, in the processing system:
a) generating procedure data indicative of at least one of:
   i) a time of the procedure;
   ii) a date of procedure;
   iii) a procedure type;
   iv) any procedure details;
   v) an indication of one or more individuals performing the procedure;
   vi) an indication of a patient; and,
b) recording an association between the procedure data and the instrument data for each instrument involved in the procedure.

Typically the event includes creating a tray of instruments for use in a medical procedure, and wherein the method includes, in the processing system:
a) determining tray data indicative of at least one of:
   i) a time of the tray is created;
   ii) a date of the tray is created; iii) an indication of one or more individuals creating the tray;
   iv) a procedure type associated with the respective tray;
   v) any procedure details;
   vi) an indication of one or more individuals performing the procedure;
vii) an indication of a patient; and,
b) recording an association between the tray data and the instrument data included on the tray.

Typically the method includes, in the processing system:
a) determining a list data indicative of a list of available instruments;
b) displaying a list of instruments for inclusion on a tray, the list being based on the list of available instruments; and,
c) using the received indicating data to identify the instrument selected for inclusion on the tray.

Typically the method includes, in the processing system:
a) determining one or more rules associated with the tray;
b) comparing the instrument data for each instrument to the one or more rules; and,
c) determining if the instrument can be included on the tray using the results of the comparison.

Typically the method includes, in the processing system:
a) determining from the instrument data, an association with at least one of:
   i) corresponding tray data;
   ii) corresponding procedure data;
   iii) corresponding batch data; and,
b) accessing the corresponding data using the determined identifier; and,
c) performing one or more actions with the corresponding data.

Typically the method includes, in the processing system, at least one of:
a) displaying the corresponding data; and,
b) locating one or more other instruments associated with the corresponding data.

Typically the code is encoded on the instrument using a laser marking process.

Typically the code is a 2-D bar code.

Typically the event includes at least one of:
a) packing an instrument;
b) repairing an instrument;
c) checking the instrument to ensure any requirements are met; and,
d) auditing instruments.

Typically the method includes, determining, using at least one of the instrument data and event data representing one or more events performed with one or more instruments, at least one of:
a) details of one or more events involving the instrument;
b) instruments involved in one or more events;
c) a schedule repair maintenance time for the instrument;
d) consumables required for performing one or more events; and,
e) cost information relating to the instruments or events.

Typically the unique identifier is based on:
a) a country code indicative of the country in which an entity marking the instrument is located;
b) an indication of the entity; and,
c) an alpha numeric code unique to the entity.

In a second broad form the present invention provides apparatus for tracking a medical instrument, the apparatus including a processing system for:
a) receiving indicating data from a scanning device, the indicating data being indicative of a unique identifier and being determined when the scanning device is used to scan the instrument;
b) obtaining, from a database and using the indicating data, instrument data associated with the instrument; and,
c) interacting with the instrument data to perform at least one of:
   i) recording one or more events performed with the instrument; and,
   ii) determining one or more events performed with the instrument.

Typically the processing system performs the method of the first broad form of the invention.

In a third broad form the present invention provides a method of laser marking an instrument for use in an instrument tracking system, the method including, in a processing system:
a) determining a unique identifier;
b) encoding the unique identifier as a machine readable code; and,
c) controlling a laser to thereby selectively expose bonding material provided on the instrument to thereby apply the code on the surface of the instrument.

Typically the instrument is mounted in a rig adjacent an aperture, and wherein the method includes, in the processing system, selectively positioning the laser to thereby position the focal spot aligned with the aperture.

Typically a number of instruments are provided adjacent respective number of apertures provided in an aperture array, and wherein the method includes, in the processing system:
a) receiving an indication of the number of apertures;
b) for each aperture, determine a unique identifier; and,
c) encode a code on each of the number of instruments.

Typically the laser includes an imaging system, and wherein the method includes, in the processing system:
a) receiving image data from the imaging system;
b) determining, using the image data, the relative position of the aperture relative to the laser; and,
c) selectively positioning the laser based on the relative position.

Typically the laser includes a bonding material application system, and wherein the method includes, applying the bonding material;
a) selectively positioning the bonding material application system adjacent an aperture; and,
b) causing the bonding material application system to apply bonding material to the instrument.

Typically the method includes:
a) receiving indicating data from a scanning device, the indicating data being indicative of the unique identifier and being determined when the scanning device is used to scan the code; and,
b) confirming the unique identifier matches the identifier used to generate the code, thereby confirming the machine readability of the code.

Typically the method includes, in the processing system:
a) generating instrument data indicative of the instrument; and,
b) recording an association between the unique identifier and the instrument data.

Typically the method further includes, in the processing system:
a) receiving indicating data from a scanning device, the indicating data being indicative of the unique identifier and being determined when the scanning device is used to scan the tag;
b) obtaining, from a database and using the indicating data, the instrument data associated with the instrument;
c) displaying at least some of the instrument data; and,
d) receiving, via an input device, input commands confirming that the instrument has been correctly identified; and,
e) making the instrument available for use following a successful confirmation.

Typically the method includes determining the unique identifier by at least one of:
a) obtaining the unique identifier from another processing system; and,
b) generating the unique identifier using a predetermined algorithm.

In a fourth broad form the present invention provides apparatus for use in laser marking an instrument for use in an instrument tracking system, the method including, in a processing system for:
a) determining a unique identifier;
b) encoding the unique identifier as a machine readable code;
c) controlling a laser to thereby selectively expose bonding material provided on the instrument to thereby apply the code to the surface of the instrument.

In a fifth broad form the present invention provides apparatus for use in laser marking a medical instrument, the apparatus including:
a) a frame having at least one aperture therein;
b) at least one support member for urging the medical instrument against the frame, the medical instrument having provided thereon a marking area, and the marking area being aligned with the aperture.

Typically the support member is formed from:
a) a shaft;
b) a housing;
c) a mounting pad coupled to the housing; and
d) a resilient member for urging the mounting pad towards the frame, to thereby urge the medical instrument against the frame.

Typically the mounting pad is pivotally mounted to the housing.

Typically the apparatus includes:
a) a frame;
b) a marking system mounted to the laser frame and arranged so as to expose the instrument, through the aperture, using a radiation beam; and,
c) a controller for controlling the radiation beam to thereby apply the code to the instrument.

Typically the apparatus includes:
a) a laser for generating a laser beam; and,
b) an optical system for focussing the laser beam into a marking area aligned with the aperture.

Typically the laser is a CO₂ laser, and wherein the optical system is a high power density focusing optics system.

Typically the marking system includes an imaging system, and wherein the imaging system is adapted to generate an image used in aligning the radiation beam with a selected one or the apertures.

Typically the code is applied to a bonding material.

Typically the marking system includes bonding material application system for applying the bonding material to the instrument.

Typically the bonding material application system is formed from a printing system.

Typically the marking system includes a scanning device for:
a) scanning a code provided on an instrument; and,
b) providing indicating data indicative of the unique identifier, to a processing system.

In a sixth broad form the present invention provides a method of tracking assets within a medical environment, the method including, in a processing system, determining an association between patient data indicative of a patient involved in a medical procedure and asset indicating data indicative of assets used within the medical environment.

Typically the method includes, in the processing system:
a) determining assets to be associated with the patient during one or more procedures; and.
b) recording an association between the patient data and the asset indicating data.

Typically the method includes, in the processing system:
a) determining a procedure in which the patient is involved; and,
b) determining the assets used in the procedure.

Typically the assets include any one of:
a) inventory used in the medical procedure, the asset indicating data being at least partially based on inventory data; and,
b) instruments used in the medical procedure, the asset indicating data being at least partially based on inventory data.

Typically the asset indicating data includes asset data representing a respective asset type, the asset data being associated with at least one of:
a) inventory data representing specific inventory instances; and,
b) instrument data representing specific instrument instances.

Typically the method includes, in the processing system, tracking the supply of assets by:
a) determining, using the patient data, a procedure to be performed;
b) determining, using at least one of procedure data and preference card data, assets required to perform the procedure; and,
c) ordering assets in accordance with the determination.

Typically the method includes, in the processing system, tracking the supply of assets by:
a) receiving supply data indicative of items to be supplied to an organisation;
b) receiving indicating data from a scanning device, the indicating data being indicative of an identifier associated with each item;
c) comparing the indicating data to the supply data to ensure items are supplied; and,
d) at least one of:
   i) in response to an unsuccessful determination, arranging for supply of missing items; and,
   ii) in response to a successful comparison, updating stock data to reflect that the item is available for use.

Typically the method further includes, in the processing system:
a) determining an asset is required for use; and,
b) modifying the stock data to reflect that the item is no longer available for use.

Typically the method further includes, in the processing system:
a) comparing the stock data to one or more predetermined criteria; and,
b) ordering additional items in accordance with the results of the comparison.

Typically the method includes, in the processing system:
a) determining preference data indicating preferred items for use in a procedure;
b) using the preference data to perform at least one of:
   i) modifying the stock data to reflect that the item is no longer available for use.
   ii) ordering additional items in accordance with the results of the comparison.

Typically the method includes, in a processing system, tracking items used in a medical procedure, the method including, in a processing system:
a) receiving indicating data indicative of items used in the medical procedure; and,
b) display a list of used items, the list being used to allow medical personnel to check used items are accounted for.

Typically the method includes, in the processing system:
a) determining an item list, the item list indicating items available for use in a respective medical procedure;
b) displaying a list of available items in accordance with the determined item list; and,
c) receiving input commands, the input commands forming the indicating data.

Typically the method includes, in the processing system, displaying the list of used items by modifying the list of available items to indicate an item has been used.

Typically the method includes, in the processing system, determining the item list from at least one of preference card data and procedure data.

Typically the method includes, in the processing system:
a) determining a list of actions;
b) display the list of actions;
c) receiving indicating data indicative of completed actions; and,
d) modifying the list of actions to indicate actions have been completed.

Typically the method includes, in the processing system, determining items have been accounted for before modifying the list of actions.

Typically the method further includes, in the processing system, tracking patients by:
a) associating the patient data with a unique identifier; and,
b) causing the unique identifier to be provided on a wearable device, the wearable device being worn by the patient such that scanning of the unique identifier allows the patient data to be subsequently displayed.

Typically the method further includes, in the processing system, tracking patients by:
a) receiving indicating data, the indicating data being indicative of a unique identifier encoded on a wearable device worn by the patient, and being received from a scanning device used to scan the unique identifier;
b) accessing, using the indicating data, patient data including at least an indication of one or more health status indicators; and,
c) at least one of:
   i) displaying the patient data; and,
   ii) updating the patient data.

Typically the method further includes, in the processing system, determining assets used in procedures performed on a patient by:
a) receiving the patient data; and,
b) determining asset indicating data using the association; and
c) determining the assets using the asset indicating data.

In a seventh broad form the present invention provides apparatus for tracking assets within a medical environment, the apparatus including a processing system for determining an association between patient data indicative of a patient involved in a medical procedure and asset indicating data indicative of assets used within the medical environment.

In an eighth broad form the present invention provides a method of scheduling procedures to respective locations, the method including, in a processing system:
a) displaying, for each of a number of different locations, a schedule including an indication of procedures to be performed and an expected procedure duration;
b) receiving indicating data indicative of the status of a procedure;
c) generating an indication if the expected procedure duration is to be exceeded; and,
d) modifying the schedule in accordance with input commands from a user.

Typically the method includes, in the processing system, determining the procedure status from procedure data the procedure data being indicative of a list of actions to be performed, and actions which have been completed.

Typically the displayed schedule includes a number of time slots and an indication of one or more procedures associated with one or more of the number of time slots.

Typically the input commands correspond to dragging a procedure from first time slot to a second time slot.

Typically the first time slot is associated with a first location and the second time slot is associated with a second location.

Typically the method includes, in the processing system:
a) displaying a list of procedures to be performed; and,
b) scheduling procedures to be performed by moving procedures from the list to one of the time slots.

In a ninth broad form the present invention provides apparatus for scheduling procedures to respective locations, the apparatus including a processing system for:
a) displaying, for each of a number of different locations, a schedule including an indication of procedures to be performed and an expected procedure duration;
b) receiving indicating data indicative of the status of a procedure;
c) generating an indication if the expected procedure duration is to be exceeded; and,
d) modifying the schedule in accordance with input commands from a user.

In a tenth broad form the present invention provides a method of ordering items, wherein the method includes, in a processing system:
a) generating order data indicative of items to be supplied;
b) transferring the order data to a supplier processing system, the supplier processing system being responsive to the order data to:
   i) determine items available for supply;
   ii) cause the available items to be supplied; and,
   iii) generate supply data representing the available items;
c) receiving the supply data; and,
d) updating stock data representing the supplied items.

Typically the supply data includes an indication of items that cannot be supplied, and wherein the method includes, in the processing system: generating order data indicative of at least one of:
a) alternative items to be supplied;
b) items to be supplied from another supplier.

Typically the method includes, in the processing system:
a) receiving indicating data from a scanning device, the indicating data being indicative of an identifier associated with each item; and,
b) comparing the indicating data to the supply data to ensure items are supplied; and,
c) at least one of:
   i) in response to an unsuccessful determination, arranging for supply of missing items; and,
   ii) in response to a successful comparison, updating the stock data.

Typically the method includes, in the processing system:
a) determining preference data indicating preferred items for use in a procedure;
b) using the preference data to perform at least one of:
   i) modifying the stock data to reflect that the item is no longer available for use.
   ii) ordering additional items in accordance with the results of the comparison; and,
   iii) providing items for use in the procedure.

Typically the method includes, in the processing system:
a) determining the supplied items; and,
b) causing payment to be provided for the supplied items.

Typically the method includes, in the processing system, determining the supplied items based at least partially on at least one of the supply data and the order data.

Typically the method includes, in the processing system:
a) determining payment requirements from the supply data; and,
b) arranging for the payment to be made.

In an eleventh broad form the present invention provides apparatus for ordering items, the apparatus including a processing system for:
a) generating order data indicative of items to be supplied;
b) transferring the order data to a supplier processing system, the supplier processing system being responsive to the order data to:
   i) determine items available for supply;
   ii) cause the available items to be supplied; and,
   iii) generate supply data representing the available items;
c) receiving the supply data; and,
d) updating stock data representing the supplied items.

In a twelfth broad form the present invention provides a method of processing orders for items, the method including, in a supplier processing system:
a) receiving order data indicative of items to be supplied from an ordering processing system;
b) determine items available for supply;
c) cause the available items to be supplied;
d) generate supply data representing the available items; and,
e) transferring the supply data to the ordering processing system to allow the ordering processing system to update stock data representing the supplied items.

Typically the method includes, in the supplier processing system, and for items unavailable for supply:
a) determining alternative items to be supplied; and,
b) generating the supply data representing the alternative items.

In a thirteenth broad form the present invention provides apparatus for processing orders for items, the method including, in a supplier processing system:
a) receiving order data indicative of items to be supplied from an ordering processing system;
b) determine items available for supply;
c) cause the available items to be supplied;
d) generate supply data representing the available items; and,
e) transferring the supply data to the ordering processing system to allow the ordering processing system to update stock data representing the supplied items.

In a fourteenth broad form the present invention provides a method of tracking supply of items for use in a medical environment, the method including, in a processing system:
a) receiving supply data indicative of items to be supplied to an organisation;
b) receiving indicating data from a scanning device, the indicating data being indicative of an identifier associated with each item;
c) comparing the indicating data to the supply data to ensure items are supplied; and,
d) at least one of:
   i) in response to an unsuccessful determination, arranging for supply of missing items; and,
   ii) in response to a successful comparison, updating stock data to reflect that the item is available for use.

In a fifteenth broad from the present invention provides a method of tracking items used in a medical procedure, the method including, in a computer system:
a) receiving indicating data indicative of items used in the medical procedure; and,
b) display a list of used items, the list being used to allow medical personnel to check used items are accounted for.

In a sixteenth broad from the present invention provides a method of tracking patients, the method including, in a computer system:
a) generating patient data, the patient data including at least an indication of one or more health status indicators;
b) associating the patient data with a unique identifier; and,
c) causing the unique identifier to be provided on a wearable device, the wearable device being worn by the patient such that scanning of the unique identifier allows the patient data to be subsequently displayed.

In a seventeenth broad from the present invention provides a method of tracking patients, the method including, in a computer system:
a) receiving indicating data, the indicating data being indicative of a unique identifier encoded on a wearable device worn by the patient, and being received from a scanning device used to scan the unique identifier;
b) accessing, using the indicating data, patient data including at least an indication of one or more health status indicators; and,
c) at least one of:
   i) displaying the patient data; and,
   ii) updating the patient data.

Typically each of the broad forms of the invention may be used individual or in combination with any other broad form of the invention.

### BRIEF DESCRIPTION OF THE DRAWINGS

An example of the present invention will now be described with reference to the accompanying drawings, in which:—

FIG. 1 is a flow chart outlining an example of a process of tracking medical instruments;

FIG. 2A is a schematic side view of an example of a mounting rig for use in marking medical instruments;

FIG. 2B is a cross sectional view of the mounting rig of FIG. 2A;

FIG. 2C is a schematic plan view of the mounting rig of FIG. 2A;

FIG. 2D is a schematic side view of an example of a support member;

FIG. 2E is a schematic end view of the support member of FIG. 2D;

FIG. 3 is a schematic plan view of an example of a system for marking medical instruments;

FIG. 4A is a schematic side view of an example of a scanner;

FIG. 4B is a schematic end view of the scanner of FIG. 4A;

FIG. 4C is a schematic side view of the scanner of FIG. 4A with an attached shroud;

FIG. 4D is a schematic perspective view of the shroud of FIG. 4C;

FIG. 5 is a schematic diagram of an example of a distributed architecture for use in marking medical instruments;

FIG. 6 is a schematic diagram of an example of the base station of FIG. 5;

FIG. 7 is a schematic diagram of an example of one of the end stations of FIG. 5;

FIGS. 8A and 8B are a flow chart of an example of the process for marking medical instruments;

FIGS. 9A and 9B are examples of a user interface used in the process of marking medical instruments;

FIG. 9C is an example of a 2-D bar code used in marking medical instruments;

FIGS. 10A to 10D are a flow chart of an example of the process of using marked medical instruments;

FIG. 11 is an examples of a user interface used in the process of using marked medical instruments;

FIGS. 12A and 12B are a flow chart of an example of a stock management process;

FIG. 13 is a flowchart of an example of the process of maintaining patient data;

FIG. 14 is a flowchart of an example of the stages involved in a patient procedure;

FIG. 15 is a flow chart of an example of the process of scheduling procedures;

FIGS. 16A and 16B are schematic examples of a GUI used in scheduling procedures;

FIG. 17 is a flow chart of an example of the process of item tracking during a procedure; and,

FIGS. 18A and 18B are a flow chart of an example of the process of monitoring a procedure.

### DETAILED DESCRIPTION OF THE PREFERRED EMBODIMENTS

### Instrument Identification

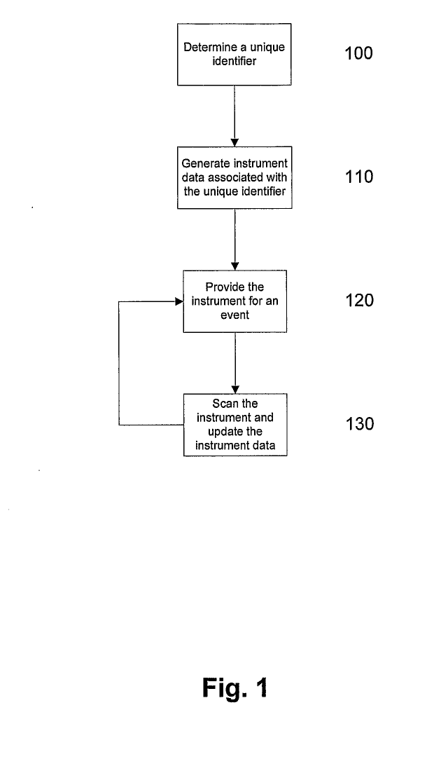

An example of a process for identifying medical instruments will now be described with reference to FIG. 1.

In particular, at step 100 a unique identifier is determined. This can be achieved in a number of ways as will be outlined in more detail below. For example, this can be achieved by generating a unique identifier which is then encoded as a machine readable code, which may be of any form such as a 2-D barcode, or the like. The machine readable code can then be applied to the medical instrument, which again may be achieved in a number of ways, as described in more detail below.

Alternatively, the identifier can be determined by imaging the instrument, for example using an infra-red imaging device, and then using the resulting image to generate the unique identifier. Thus, for example, the infra-red image may be representative of the particle structure of all or part of the instrument. As this structure is unique to the instrument, this effectively acts as a fingerprint, so that when a predetermined algorithm is applied to the image, this results in the generation of a unique number that then forms the unique identifier.

At step 110 instrument data is associated with the unique identifier and stored in a database or the like.

At step 120 the instrument is provided for an event which may include for example use in a procedure, sterilisation, reconditioning, repair or the like. At step 130 the instrument data associated with the instrument is updated to reflect the event that has occurred. The process may then be returned to step 120 allowing the instrument to be reused as required. These steps may alternatively be interchanged, so that the instrument data is updated either prior to or as part of the event.

By maintaining instrument data associated with the unique identifier, this allows the use of each instrument to be uniquely tracked, thereby allowing the use history to be subsequently reviewed as required. This therefore effectively establishes an audit trail of use for each instrument.

Thus, by including suitable information within the instrument data, such as details of the procedure performed, the date on which the procedure was performed and the details of any medical personnel involved therein, it will be appreciated that this allows for subsequent and rapid identification of any events in which the instrument has been used.

In this case, each time the instrument is used, it can be scanned to determine the unique identifier, either from the applied machine readable code, or by rescanning the instrument with an infra-red scanner to recreate the image, derive the structure fingerprint and regenerate the unique identifier using the same predetermined algorithm.

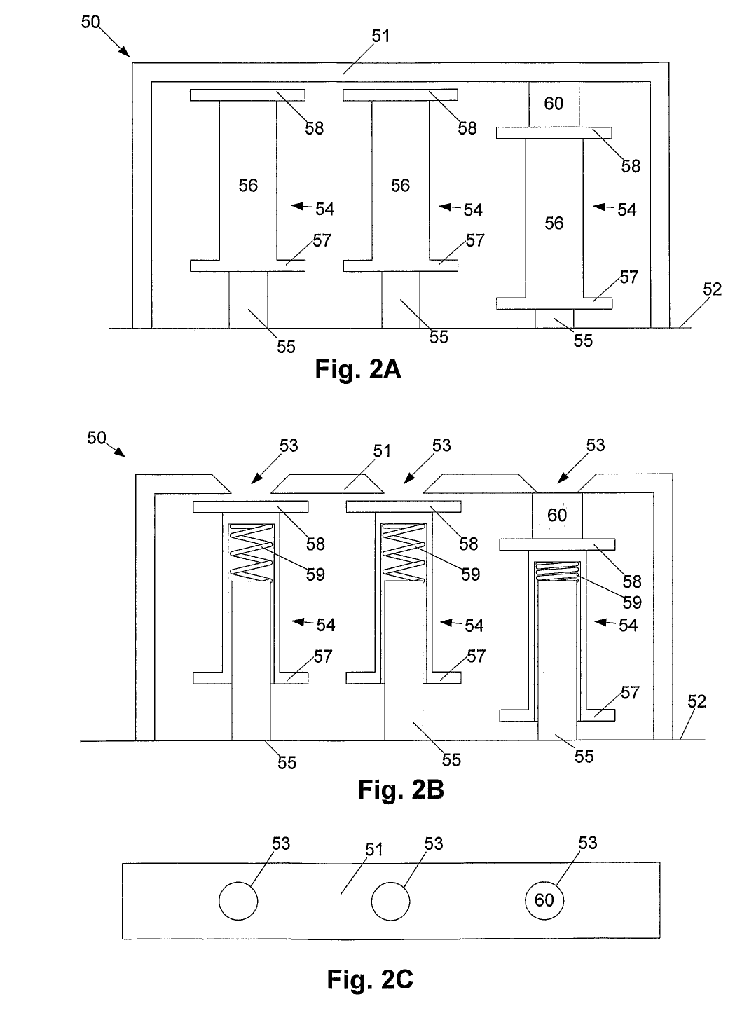

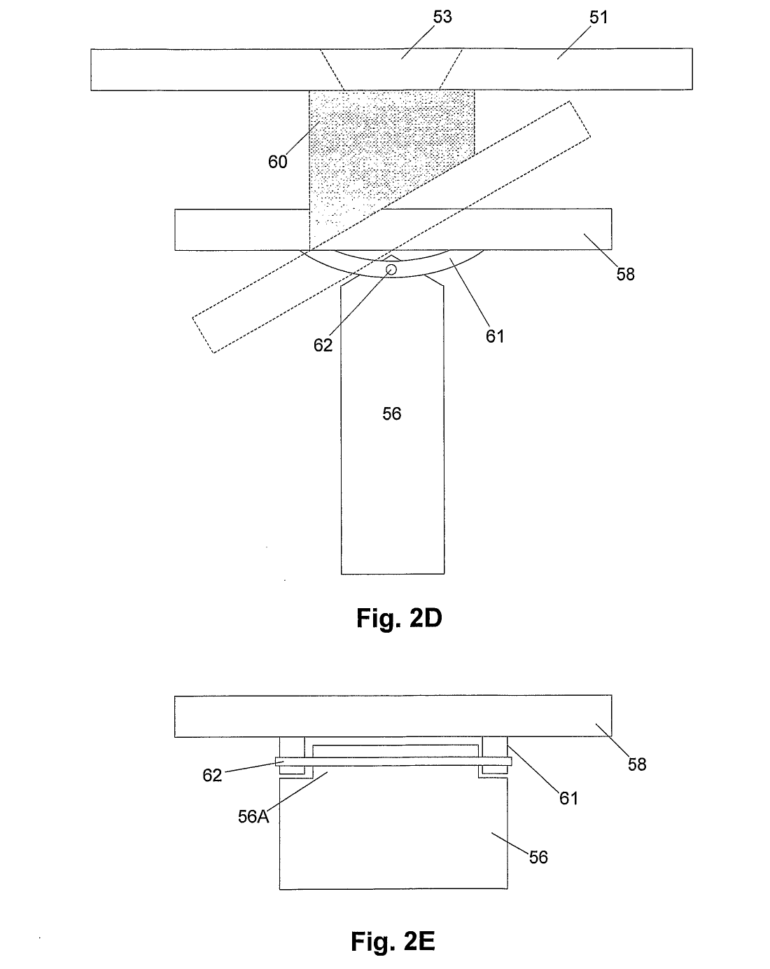

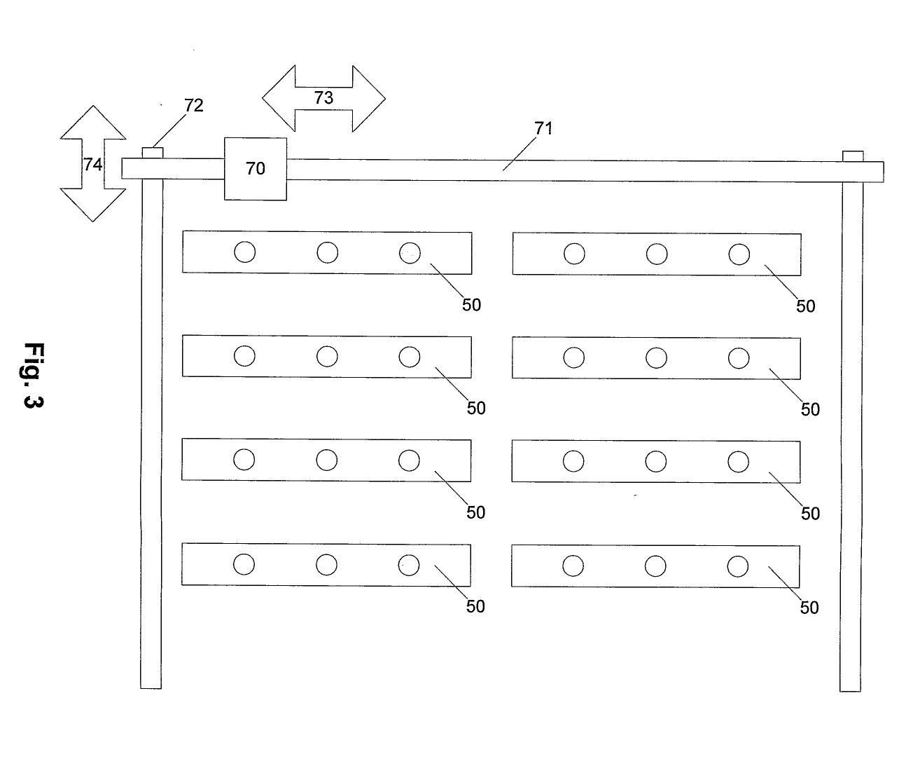

For situations in which a machine readable code is applied to the instrument, it is typical to use specific apparatus to ensure correct marking is achieved. An example of such apparatus will now be described with reference to FIGS. 2A to 2E.

In particular, as shown the apparatus used in marking the instrument includes a mounting rig 50 formed from a frame 51 provided on a base 52. The frame 51 includes a number of apertures 53, which are generally aligned with corresponding support members 54. Thus, as shown a respective support member 54 is associated with each aperture 53.

Each support member 54 is formed from a shaft 55, mounted to the base 52, and a housing 56. The housing 56 is in the form of a cylinder that sits over the shaft 55, with a spring 59 being provided between the shaft 55 and the housing 57, as shown. The housing 56 includes a skirt 57 and is coupled to a mounting pad 58.

The mounting pad 58, which is shown in more detail in FIGS. 2D and 2E, includes two arms 61 connected to an upper portion 56A of the housing 56, via an axel 62. In use, the mounting pad 58 is pivotally mounted to allow the mounting pad 58 to rotate between the extreme positions shown by the dotted lines in FIG. 2D.

In use, the housing 56 can be urged in a downward direction towards the base 52 by exertion of appropriate pressure on the skirt 57. An instrument 60 can then be placed on the mounting pad and the pressure on the skirt 57 released, thereby causing the instrument 60 to be urged against the underside of the frame 51, aligned with one of the apertures 53, as shown. It will be appreciated that as the mounting pad 58 is pivotally mounted to the housing 56, the mounting pad will rotate to accommodate the shape of the instrument as shown in FIG. 2D. This ensures that the instrument 60 is securely mounted and is unable to move, allowing it to be accurately marked.

Marking is achieved by laser marking a material applied to the instrument 60, in a process known as laser bonding.

Laser bonding is an additive process in which a material is bonded to the instrument using an Nd:YAG, YVO₄, or CO₂ laser. This can be achieved using proprietary materials, which generally consist of a glass frit powder or ground metal, oxides mixed with inorganic pigment, and a liquid carrier (usually water). Such bonding materials are available from companies such as Cerdec Corporation™ The material can be applied to the instruments using a number of different techniques depending on the preferred implementation, and this many include for example painting, spraying or printing the material directly onto the instrument surface. Adhesive backed tapes coated with an additive can also be used in this process.

In order to ensure correct marking it is important to ensure that the bonding material is provided at the focal point of the laser beam. This is achieved through the use of the mounting rig 50, with the bonding material being positioned aligned with the aperture 53, so that this ensures correct exposure of the bonding material to the laser beam, and in turn ensures accurate marking of the instrument.

Furthermore, by providing an adhesive backed dot as the bonding material, this provides a target on which the laser may align, using a suitable tracking system, thereby further enhancing the accuracy of the encoding procedure.

In general, laser bonding is preferably achieved using a YAG laser as this generally has improved optical characteristics and can therefore achieve an improved resolution compared to other laser systems. However, in this instance, the accurate alignment of the bonding material with the laser focal point, as well as the use of a consistent bonding material, allows suitable resolution to be achieved using a cheaper CO₂ laser, combined with an HPDFO (High Power Density Focusing Optics) lens system.

An example of a laser based marking system is shown in FIG. 3.

As shown the apparatus includes a number of mounting rigs 50 generally aligned in an array. Although any number may be used, eight mounting rigs 50 are shown in this example for clarity purposes only.

In addition to the array, a laser system 70 is provided on a movable frame shown generally at 71, 72. Whilst this may be achieved in any one of a number of manners, in this example the laser system 70 moves relative to the frame 71, in the direction shown by the arrow 73, whilst the frame 71 moves relative to the frame 72, as shown by the arrow 74. This allows the position of the laser system 70, with respect to the mounting rigs 50, to be controlled, thereby allowing the focal spot of the laser beam to be aligned with the apertures 53.

It will therefore be appreciated that motion of the laser system is typically achieved through the use of suitable control motors such as stepper or servo motors. This allows the position of the laser system 70 to be accurately controlled by a suitable control system, such as a computer system or the like as will be described in more detail below. This in turn allows a number of instruments 60 to be provided in the mounting rig array, and then marked in a substantially automated process under computer control.

The laser system includes a CO₂ laser with HPDFO focusing system, and as such systems are proprietary, this will not be described in further detail. The laser system may also incorporate a number of additional elements, such as a printer system for applying the bonding material to the instrument, a dryer for drying the bonding material, and a scanner for scanning the machine readable code, as will be described below.

Utilising a laser bonding process has a number of advantages over traditional marking techniques such as engraving. In particular, during the laser exposure process the bonding material bonds to the surface of the instrument, and is selectively coloured as it is exposed to the laser beam, thereby allowing 2-D markings to be applied to the surface. The bonding material is substantially level relative to the surface of the instrument, and therefore does not provide any surface relief structure which can allow material to become trapped on the surface of the instrument. The use of a bonding material ensures that the properties of the instrument, such as the strength of the instrument, are unaffected. Furthermore, by using the same bonding material on each instrument, and accurately aligning the bonding material with the laser, this allows a cheaper CO₂ based laser system to be used as opposed to a more expensive YAG laser.

In order to detect the 2-D code applied to the instrument surface, a laser based scanning system can be used. However, laser scanning of medical instruments is generally hampered by the fact that the instrument surface is highly reflective.

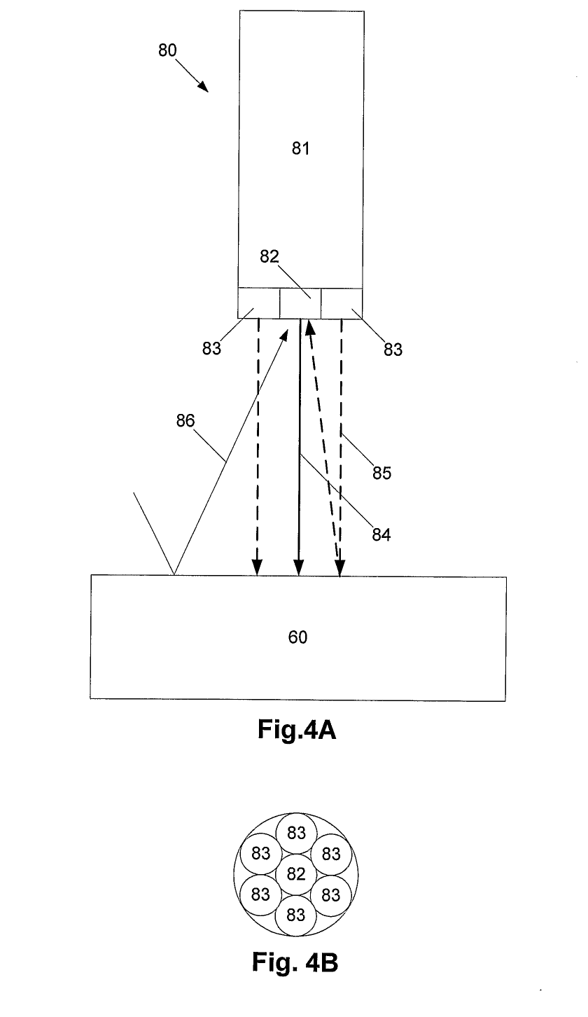

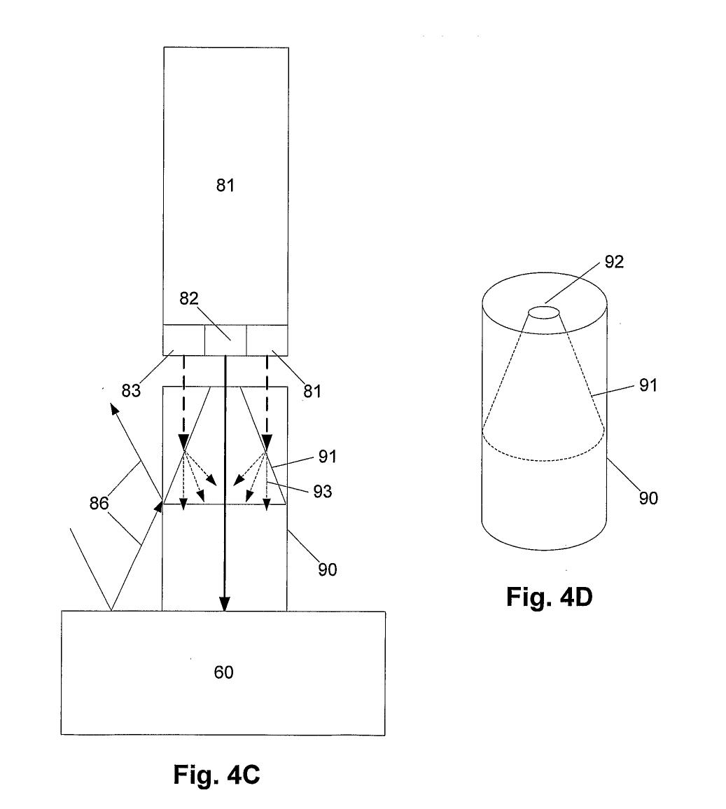

An example of a scanner utilised to scan barcodes is shown for example in FIGS. 4A and 4B. As shown the scanner 80 includes a body 81 having a laser emitter/detector element 82 and a number of illumination sources 83.

In use, the laser emitter/detector element 82 generates a laser beam shown generally at 84, which exposes an area of the surface of the instrument 60, typically using a raster pattern or the like. The laser emitter/detector element 82 detects reflected radiation, and uses this to sense the barcode or other 2-D code applied to the instrument. The barcode will typically be interpreted by suitable processing provided in the housing 81, allowing an output indicative of the barcode (or the information encoded therein) to be output, as will be appreciated by persons skilled in the art.

In addition to this, a number of illumination sources 83 are arranged surrounding the laser emitter/detector element 82 to allow the surface of the instrument to be illuminated. This helps to ensure even illumination over the surface of the instrument 60, which in turn aids with the scanning process.

Such instruments are known in the art and an example of one is Code Corp™s Code Reader 2.0 (CR2) device.

However, such instruments suffer from a number of drawbacks. In particular, stray reflections from the surface of the instrument 60, as shown at 86 can impinge upon the laser emitter/detector element 82, and interfere with the detection of the reflected laser beam 84. This problem is exacerbated in many cases by the presence of glass shield (not shown) provided over the scanning element, with reflections onto the glass shield causing flaring and internal reflections. This, in turn, affects the results of the scanning. Similarly, radiation from the illumination sources, as shown generally at 85, can also be reflected from the instrument surface to thereby cause similar problems.

Accordingly, the scanner is preferably modified by the utilisation of a shroud shown in FIGS. 4C and 4D. The shroud includes an outer housing 90 having a reflective inner surface, and a diffuser cone 91, having an opening 92 therein.

In use, when the housing 90 is coupled to the scanner 80, the opening 92 is aligned with the laser emitter/detector element 82, allowing the laser beam 84 to pass through the opening 92 unimpeded. This allows the laser beam 84 to expose the surface of the instrument 60 in the normal way. However, the presence of the housing 90 prevents stray reflections 86 from impinging on the scanning element 81 as shown.

In addition to this, the diffuser cone 91 diffuses radiation from the LEDs 82, as shown by the arrows 93. The diffused radiation is reflected from the inner surface of the housing 90 and impinges on the surface of the instrument 60. By diffusing the radiation and reflecting it from the inner surface of the housing 90, this ensures that the radiation is incident on the instrument 60 at a range of angles substantially over the entire region surrounding the 2-D code being detected.

This helps reduce the chance of stray reflections impinging on the laser emitter/detector element 82, and provides a constant intensity of background radiation reflected from the entire instrument surface 60. This in turn allows the laser emitter/detector element 82 to distinguish the reflected radiation beam from the background radiation thereby ensuring accurate scanning of the code provided on the instrument 60.

It will be appreciated that a similar scanning device incorporating an infra-red laser may be used to generate the infra-red image outlined above.

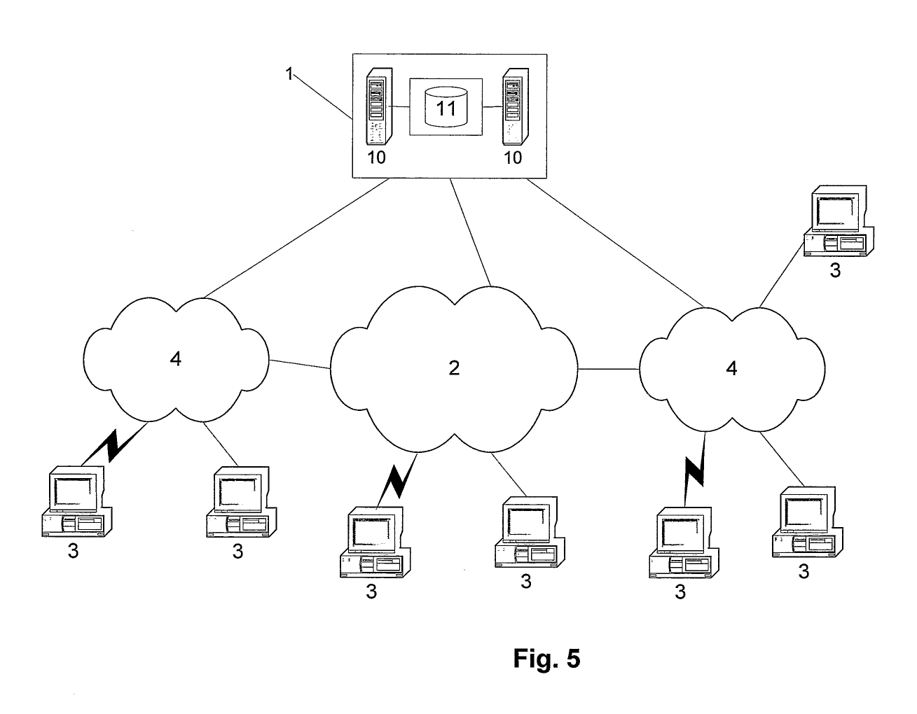

Apparatus suitable for administering instrument marking will now be described with respect to FIGS. 5 to 7.

As shown the apparatus includes a base station 1 coupled to a number of end stations 3 via suitable communications networks 2, 4, such as the Internet and/or one or more Local Area Networks (LANs), Wide Area Networks (WANs), or the like. The base station 1 typically includes a processing system 10 coupled to a database 11 as shown.

In one example, the base station 1 operates to control the creation and distribution of unique identifiers, thereby allowing instruments to be uniquely marked on a global basis. The unique identifiers can be obtained by users of the end stations 3, and then applied to instruments allowing the instruments to be tracked.

It will therefore be appreciated from this that the end stations 3 are adapted to communicate with the base station 1, utilising appropriate communications techniques, thereby allowing unique identifiers to be generated and assigned by the base station 1 and then returned the end station 3. It will therefore be appreciated that a wide range of architectures may be used, and that the current example is for the purpose of illustration only.

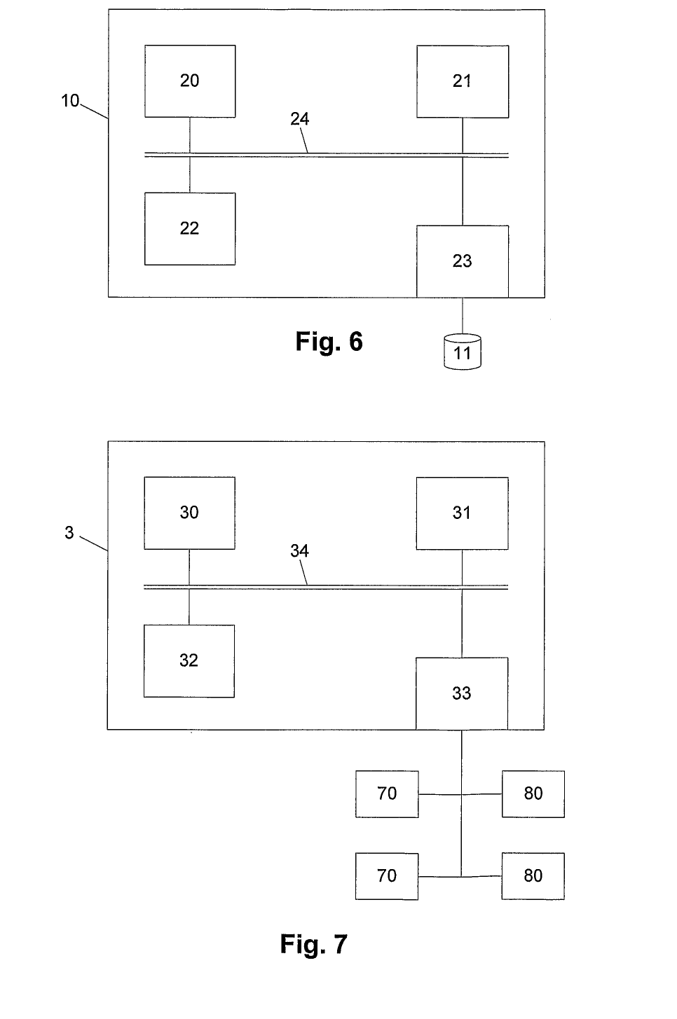

An example of a suitable processing system 10 is shown in FIG. 6. As shown the processing system 10 includes a processor 20, a memory 21, an input/output device 22, such as a keyboard and display or the like, and an external interface 23, coupled together via a bus 24. In use the external interface 23 may be coupled to the database 11, as well as providing connections to the communications networks 2, 4. Accordingly, the processing system 10 may be any form of processing system, such as a computer server, a network server, a web server, a desktop computer, a lap-top or the like. Alternative specialised hardware may be used.

Similarly, the end station 3 may be formed from a processor 30, a memory 31, an input/output device 32 and an external interface 33, coupled together via a bus 34. Again the external interface 33 may be used to provide a connection to the communications networks 2, 4 as well as allowing the processing system 3 to be coupled to either the laser system 70 and/or one or more scanners 80, as shown. Accordingly the end station 3 may be any form of computer system such as a desktop computer, lap-top, specialised hardware or the like.

In one example, the base station 1 and the end stations 3 communicate via the Internet to allow one or more unique identifiers to be requested by the end station 3, and then delivered via the communications networks 2, 4. This may therefore be achieved through the use of a suitable web-pages hosted by the base station 1.

In this case, a user of the end station 3 will access a web-page presented by the base station 1, and select an appropriate option to request one or more unique identifiers. Typically identifiers are only supplied to authorised users of the system, and the user of the end station 3 therefore typically has to undergo an authentication procedure. This may be achieved using any one of a number of techniques, such as the use of certificates or the like, but will typically involve having the user submit a username and password, or the like, which is then authenticated by the base station 1, in the normal way. Thus, it will be appreciated from this that the user will typically have to undergo a registration procedure before being able to obtain identifiers, and hence use the tracking system.

In any event, once the user has been authorised, the base station 1 generates one or more identifiers as required. The base station 1 may generate the unique identifier based on certain information, depending on the implementation, and this may therefore require the user to provide the information when requesting the identifiers, or may use information stored in the memory 21 and/or the database 11 during a registration procedure. Additionally, the identifiers are to be unique, and accordingly, the base station 1 will typically use details of previous assigned identifiers to ensure that identifier duplication does not occur.

The identifiers may then be downloaded to the end station 3, for example, via the Internet, or supplied to the user of the end station 3 on physical media, such as a CD, DVD or the like. In either case, the identifiers are typically provided in a predetermined format, such as in a text file, CSV file or the like.

As an alternative, the base station 1 may provide software to allow a user to generate their own identifiers using the end station 3. In this instance, the software may be provided via download, or on physical media, such as a CD, DVD or the like. The software is typically adapted to generate identifiers which are specific to the respective user. To achieve this, the software can incorporate information relating to the user, so that the identifiers generated by the software are globally unique.

An example of a suitable identifier will now be described.

In particular, a number of factors need to be considered when producing the identifiers, including the maximum and minimum code sizes.

The code size will in turn affect the size of the resulting 2-D code and it is therefore necessary to ensure a maximum length is set to ensure that the resulting 2-D code can be read accurately. In particular, if the code becomes too large, then this will require either a high code density, or alternatively a code extending over a large area. In the first case, the laser encoding system and the scanner both require a high degree of accuracy that in turn requires more expensive optical and laser equipment. In the second case, the laser system and the scanner must be able to encode or read codes over a larger area, which again increases the complexity and cost. Additionally, there can be problems in providing large codes on some pieces of equipment, which may have only a small encodable surface area.

However, this must be counterbalanced by a minimum code size required to ensure that the generated identifier is globally unique.

To achieve this, in one example, the unique identifier is formed from three portions, namely:
Country code—typically derived from the ISO 3166 country codes and reflecting the location of the entity marking the instrument;
Prefix—reflecting the identity of the entity marking the instrument; and,
Unique number—a sequence of alphanumeric characters that is unique for instruments marked by the respective entity.

Thus, an example could be in the form AU BT8 JK4WT, where:
AU—country code;
BT8—prefix; and,
JK4WT —unique number.

In general, at least the country code and prefix will be scrambled or otherwise disguised so that it is not possible for the country code or prefix to be determined by third parties. Thus, whilst the country code and prefix will generally be the same for a given encoding entity, the alpha numeric characters will be effectively meaningless if the resulting code is scanned and the unique identifier decoded. This helps reduce the chance of entities fraudulently generating their own codes, as well as preventing third parties being able to determine the entity and the location in which the encoding was performed.

Similarly, it is typical for the unique numbers to be generated non-sequentially, again thereby preventing entities obtaining a single identifier, and then generating further identifiers by incrementing the provided unique number. In this instance, if the entity than also obtained further identifiers from the base station 1 they may duplicate the identifiers already generated.

Additionally, mixing the unique number with the prefix and country code, effectively scrambling the entire identifier, helps further prevent fraudulent identifier use.

It will be appreciated from this, that the marking of instruments may be performed by any one of a number of entities, depending at which stage the instrument is marked.

Thus, for example, instruments in hospitals or other medical facilities may be marked, to allow the process to be applied to existing instruments, in which case the prefix will reflect the identity of the hospital. Alternatively, a manufacturer or supplier may mark the instruments when the instruments are first created or sold.

It will be appreciated that as each entity uses it's own unique numbers, then these numbers can be generated internally using suitable applications software executed by the end station 3. In this case, the user of the end station 3 will therefore obtain the one or more identifiers by downloading the software from the base station 1. In this case, the user (or the marking entity with which the user is associated) is assigned the country code and prefix, and this can be supplied together with, or as part of the software, thereby allowing the software to generate the identifiers for the respective entity. In this case, the entity need only contact the base station a single time during initial set-up of the end station 3, allowing the end station 3 to generate the identifiers in future.

However, any unique code can be used as the unique identifier. Accordingly, in the event that the infra-red image is used to derive a unique “fingerprint” based on the infra-red image of the instrument structure, then this can simply be translated to a suitable alpha-numeric code using a suitable algorithm.

It will also be appreciated that as long as the resulting identifiers are unique then the different techniques for identifier generation can be used in conjunction within the same tracking system. Thus for example, some instruments can be marked with a machine readable code, whilst others have the unique identifier derived from the structure of the instrument via the infra-red image.

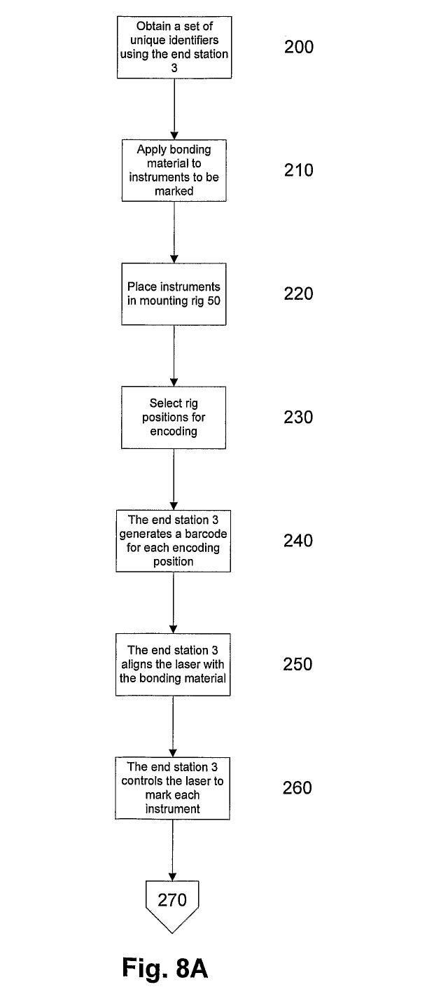

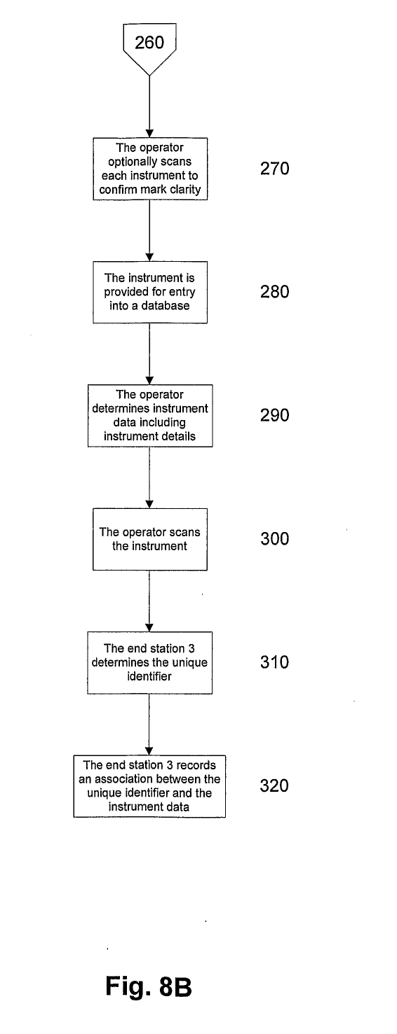

The end station 3 can then use the generated identifiers to mark instruments as will now be described with respect to FIGS. 8A and 8B.

Initially, at step 200, a user will utilise the end station 3 to obtain a set of unique identifiers as described above. At step 210 the user applies bonding material to the instruments to be marked, and then places the instruments in the mounting rigs 50, at step 220.

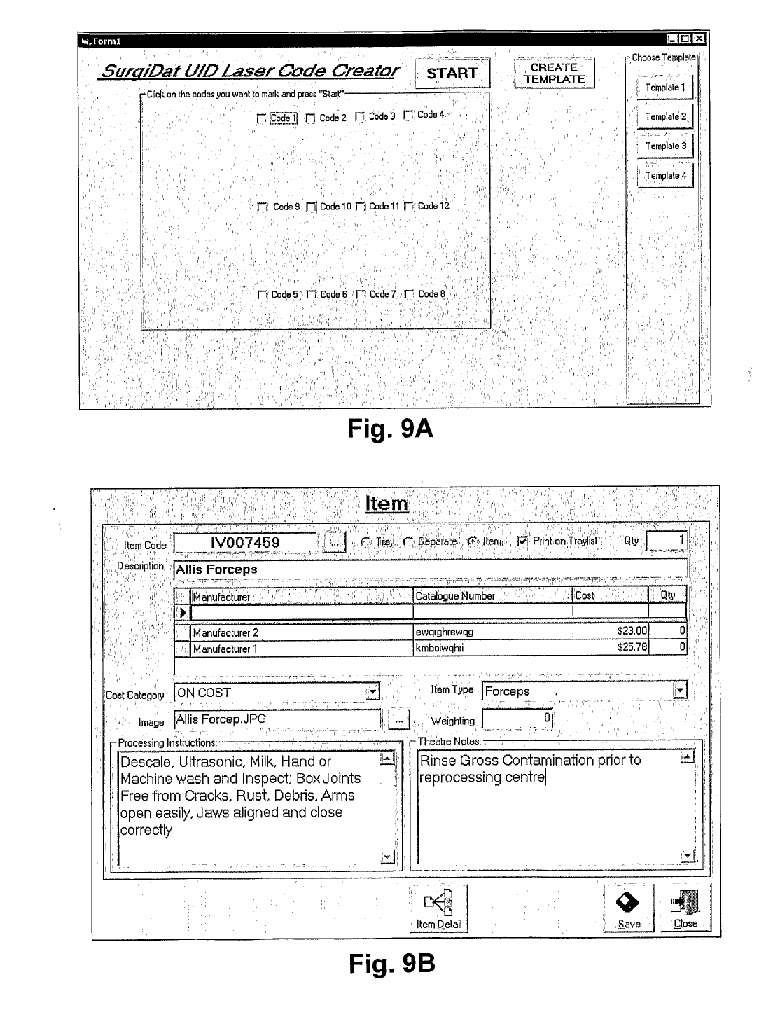

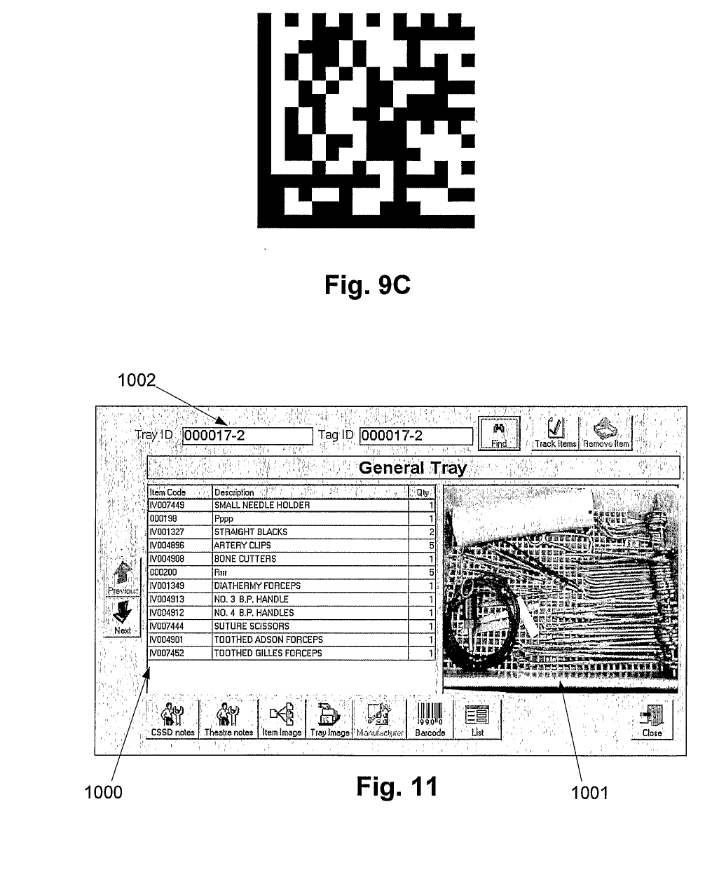

The user then executes appropriate application software provided on the end station 3 to control the marking of the instruments. The application software will provide the user with a representation of an array of mounting rigs 50 as shown for example in FIG. 9A. In this example, twelve encoding positions are shown, although it will be appreciated that this is for the purpose of example only.

The user selects positions in the representation corresponding to the location of instruments to be marked in the physical array of rigs 50, thereby selecting the rig positions for encoding, at step 230.

At step 240 the end station 3 generates a barcode for each encoding position based on a selected unique identifier. The barcode may be encoded using any one of a number of techniques and will typically utilise standard encoding algorithms available in the art. This will not therefore be described in any detail, although a representation of a typical 2-D barcode is shown in FIG. 9C.

In any event, once the end station 3 has generated the barcode, the end station 3 operates to control the laser system to cause marking of each instrument. This is achieved by having the end station 3 activate the stepper motors to control the positioning of the laser system 70 relative to the array of rigs 50, thereby aligning the exposing laser beam with the bonding material applied to the instrument mounted in a respective one of the apertures 53, at step 250.

In use, this can be achieved using any one of a number of suitable control systems. Thus, for example, the laser system 70 can be moved to a predetermined position which is aligned with the aperture. However, as an alternative, the laser system can include a suitable imaging system which is adapted to image the array, allowing the end station 3, or other control processing system, to detect the position of the bonding material. In the example in which the bonding material is applied in the form of an adhesive dot, the end station 3 will operate to locate the dot in the image, and then control the position of the laser system 70, to thereby align the laser beam with the dot.

It will be appreciated that a combination of the two techniques can also be used, to allow initial gross alignment with the array to be performed by moving the laser system 70 to a predetermined location, and then using an imaging process to perform more accurate alignment.

The end station 3 will then operate to activate the laser beam, with the beam being modulated so as to encode the corresponding barcode in the bonding material, at step 260. The modulated laser beam impinges on the bonding material and thereby operates to bond the material to the instrument surface, as well as to encode a visible representation of the barcode therein.

Once each barcode is encoded, the end station 3 controls the relative position of the laser system 70, thereby moving the laser system 70 to the next aperture 53 in which an instrument is located, thereby allowing each instrument in the array to be encoded in turn.

Once each instrument has been encoded the operator optionally scans each instrument using a scanning device 80, at step 270. The scanner 80 will generate indicating data indicative of the barcode, or the encoded unique identifier, and transfers this to the end station 3 to allow the clarity of the resulting code to be confirmed. Thus, this process ensures that the barcode can be read to determine the unique identifier.

At step 280, the instrument is then provided to allow a suitable database entry to be created. To achieve this, at step 290, the user provides instrument details for each instrument to be marked using an appropriate input screen, as shown for example in FIG. 9B. The instrument details will typically include some or all of the following information:
an instrument description;
an item code indicative of a type of the instrument;
manufacturer details;
date of purchase;
warranty details or requirements;
instrument cost;
replacement information or requirements;
instrument cleaning instructions or requirements;
processing or maintenance information or requirements;
a risk level associated with the instrument;
status information, such as whether the instrument is available for use;
equivalent instruments;
instrument use instructions or requirements; and,
a photo.

Additional information may also be provided depending on the implementation.

In any event, at step 300, the user scans the instrument, using the scanner 80, to allow the end station 3 to decode the unique identifier at step 310. At step 320, the end station 3 records an association between the unique identifier and the instrument data, such that the instrument data can then be subsequently retrieved when the instrument is scanned.

It will be appreciated that the instruments may be marked either by a manufacturer or supplier, or by an end user, depending on the respective implementation. In the former case, the instrument may be provided with no associated instrument data, thereby allowing the instrument data to be defined by the end user. However, additionally, or alternatively, the manufacturer/supplier may create instrument data to allow the instrument to be tracked through the supply chain. In this instance, the end user may create new instrument data, or use some or all of the manufacturer's instrument data, modifying this to suit their own purpose, as required. The instrument data is typically stored in a centralised repository, which may be administered by a central server or base station, such as the base station 1, as will be described in more detail below.

It will also be appreciated that in the event that the unique identifier is derived from an infra-red image of the instrument, then the marking steps 200 to 270 can be effectively omitted. In this example, steps 280 and 290 are performed to generate the database entry. At step 300 the instrument is scanned with an infra-red scanner to generate the image, which is then used to generate the unique identifier at step 310. The end station then proceeds to create the association as in the example above at step 320.

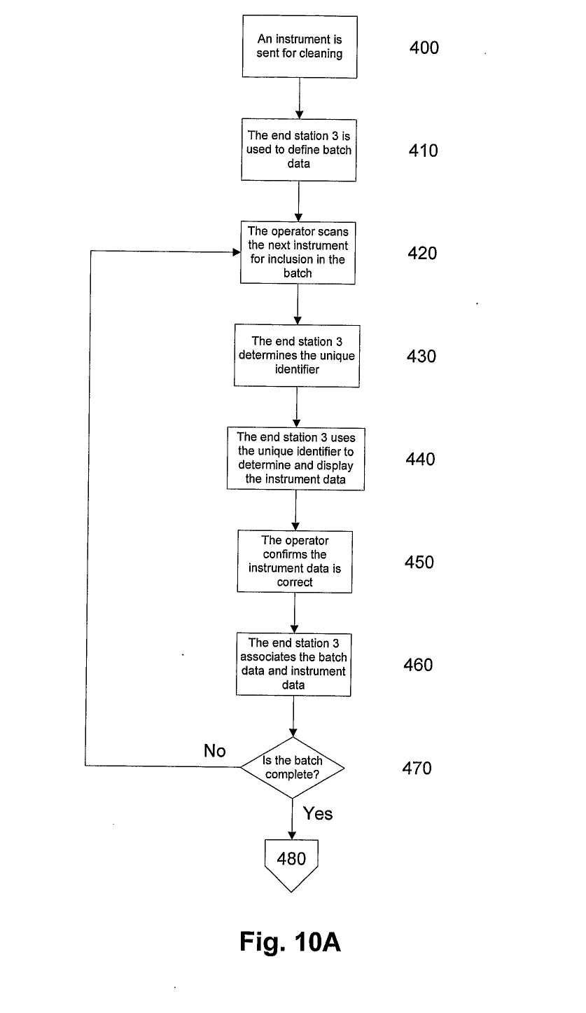

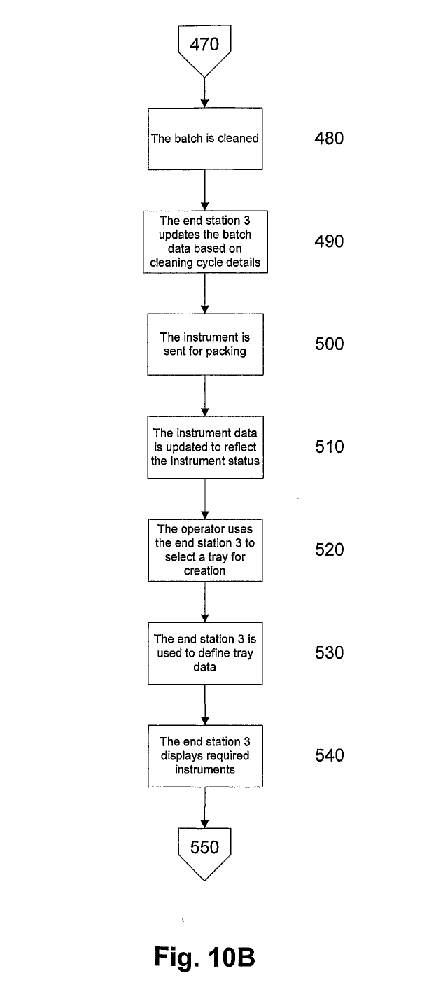

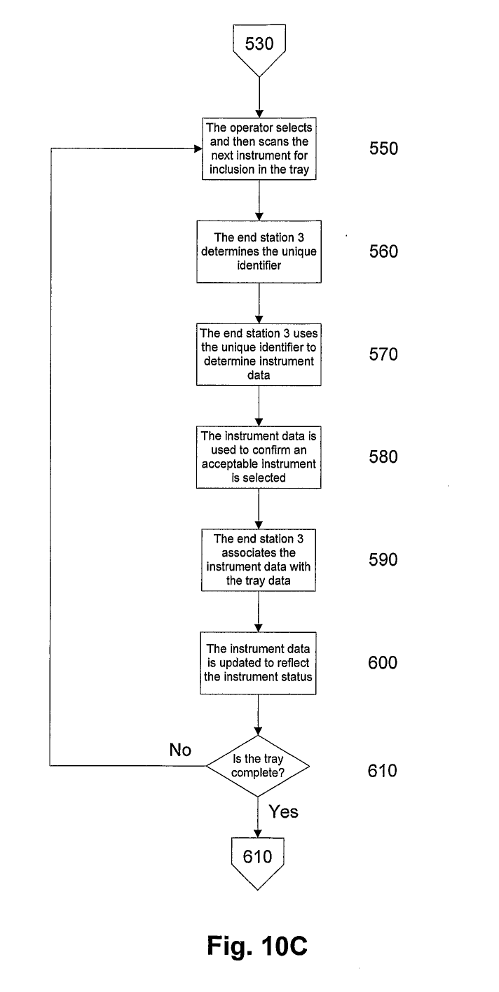

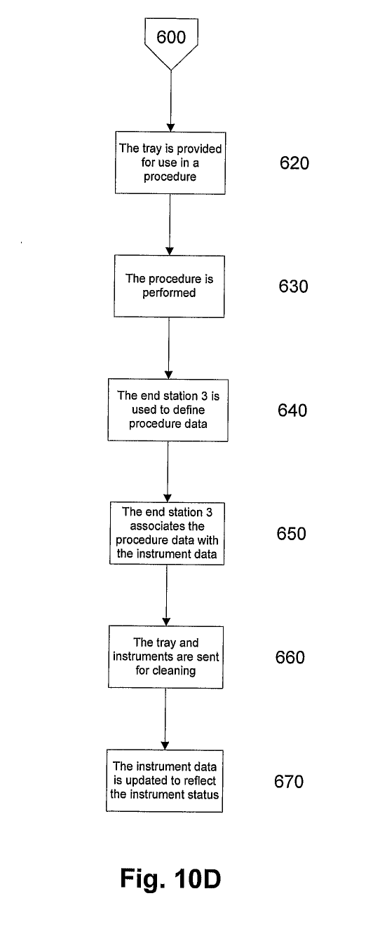

An example of the tracking of an instrument will now be described with reference to FIGS. 10A to 10D.

In particular, after an instrument has been marked, or after use, the instrument is sent for cleaning, which may include sterilisation and other appropriate cleaning techniques, at step 400. When a user is to perform cleaning, the user will typically perform this as a batch process, with a number of instruments being cleaned at the same time. Accordingly, the end station 3 is used to define batch data at step 410.

The batch data includes details of the cleaning procedure, such as:
the cleaning technique used;
the time and date on which the cleaning occurred; and,
details of the individual performing the cleaning operation.

The user will then scan the next instrument for inclusion in the batch at step 420. The end station determines the unique identifier associated with the scanned instrument at step 430, using this to determine and display the instrument data at step 440. At step 450 the user confirms the instrument data is correct before associating the batch data and the instrument data to thereby add the instrument to the assigned batch at step 460.

Associating instrument and batch data may be achieved in a number of manners depending on how the instrument data and batch data are stored. For example, the instrument and batch data may be stored in respective database tables, with each instrument or batch being provided a table row. In this case, a link can be defined between corresponding rows in the batch and instrument tables to thereby associate the instrument with the corresponding batch. Alternatively, the batch data may include an identifier which is stored as part of the instrument data, or vice versa.

At step 470 it is determined if the batch is complete, and if not, the process returns to step 420 allowing the user to scan the next instrument.

Once the batch is complete, the process moves on to step 480 to allow the batch to be cleaned. During or following this, the batch data can be updated with information regarding the cleaning process at step 490. This information can be obtained from the machine used in performing the cleaning, which is typically able to provide cycle data including details such as:
a cleaning time;
a cleaning temperature;
a cleaning pressure;
machine maintenance information;
a machine operator identifier; and,
other cleaning details.

Accordingly, subsequently scanning an instrument will allow an end station to determine the unique identifier, and consequently the instrument data associated with the instrument. From this, the corresponding batch data can be accessed, thereby allowing details of the cleaning procedure for the respective instrument to be determined.

It will be appreciated that the above described process can be performed in a number of ways, and the above is for the purpose of example only. Thus, the batch data could be created prior to, after or during the cleaning procedure. Similarly, a batch may include only a single instrument, depending on the cleaning performed, and the term batch should therefore be understood to be for the purpose of example only.

At step 500 the instrument is sent for packing or other further processing. This may include placing the instrument in packs or the like, as well as adding instruments to a tray, as will be described below.

At step 510, the end station 3 updates the status of the instrument stored as part of the instrument data to indicate that that the instrument is now available for packing.

An example of the process of adding an instrument to a tray will now be described, although it will be appreciated that the general steps outlined could also apply to other parts of the packing process.

At step 520 the user uses the end station 3 to select a tray for creation. This can be achieved using a suitable graphical user interface, so that, for example, the user of the end station 3 can be presented with a drop down list of different tray types available. The user will select a tray type to be created, causing the end station 3, causing the GUI to display details of required instruments at 1000, a photo of the completed tray 1001, and a tray ID, as shown in FIG. 11.

At step 530, the end station 3 generates tray data, which is typically achieved by adding an entry to a tray table in the database. The tray data typically includes details of the tray creation process including:
the tray ID;
the time and date on which the tray was created; and,
details of the individual creating the tray.

In the example of FIG. 11A, the tray ID, shown at 1002, includes a prefix (000017 in this example) indicative of the type of tray being created, and a suffix (−2 in this example), which is indicative of the number of trays of that type being created.

The tray data also typically includes an identifier which is determined by scanning an appropriate barcode. Accordingly, the tray may be marked using the process described above, in a similar way to other instruments. Alternatively, the tray may be identified by a tag applied to the tray, or provided on the tray for example, using a suitable label or the like.

The list of the instruments to be provided in the tray, typically includes:
an item code indicative of the instrument type; and,
a description of the instrument.

If the user then selects a respective one of the instruments from the list, additional details can be displayed, including for example, a photo of the instrument, a more detailed description of the instrument and any associated requirements or restrictions on its use.

This aids the user in selecting a suitable instrument from the available instruments. In particular, the user can request an instrument based on the required item code, which is used to obtain an instrument of the correct type from stores. The item code may also be provided on any packaging or the like, to further aid instrument identification.

The instrument is then scanned using the scanner 80 at step 540, allowing the end station 3 to determine the unique identifier at step 550, and uses this to determine corresponding instrument data at step 560.

The instrument data is used to confirm a suitable instrument has been selected. This can be achieved in a number of manners. For example, this can cause the end station 3 to display the instrument data to the user, to allow the user to view any associated restrictions on use, as well as to view the item code, to ensure the correct instrument is displayed. Additionally, or alternatively, the instrument data can be compared to the tray data, using predetermined business rules, to determine if the instrument is suitable for use.

This is required, for example, to take account risk levels associated with instruments. For example, when an instrument is used in a high risk procedure, this is indicated within the instrument data. In this instance, this may indicate that the instrument can only be used in certain procedures. Accordingly, the intended use of the tray is determined, based on the tray type indicated by the tray ID, allowing the end station to determine if the instrument is suitable for use on the corresponding tray.

The use of such a technique helps reduce the chance of instruments being incorrectly provided on certain trays, which in turn can help reduce the chance of the instruments being used incorrectly.

At step 590, the end station 3 associates the instrument data with the tray data, and updates the status provided in the instrument data and the tray data at step 600. In this example, the end station achieves this by generating a suffix to be included on the item code, to thereby create a unique item code for each instrument of that type of the tray. The unique item code for that tray is then stored in the tray data, with an association being recorded between the unique item code and the instrument data of the corresponding instrument.

This allows each instrument within a given tray to be uniquely identified, thereby allowing the use of instruments on different trays to be tracked.

If the tray is not complete, the process returns to step 550 allowing the next instrument to be selected at step 610.

Otherwise, at step, at step 620 the tray is provided for use in a procedure. The manner in which this is performed is typically in accordance with standard procedures, which are typically specific to the medical institution performing the procedure.

At step 630, the procedure is performed, with the end station 3 being used to create procedure data reflecting the details of the procedure. Typically the details of the procedure will include information such as:
the nature of the procedure;
the medical practitioners involved in performing the procedure;
the time and date of the procedure; and,
information indicative of the patient.

Due to security reasons it is typical that the patient identity is not included directly in the procedure information, but rather an identifier can be used which is associated with the identity of the patient for example by way of an appropriate mapping through the hospitals and internal secure records.

The end station 3 then operates to associate the procedure data and the instrument data. This may be achieved for example by directly associating the procedure data with the instrument data for each instrument, or by associating the procedure data with the tray data of the tray, which is in turn linked to the instrument data of the instruments provided thereon.

Once the tray has been used, the instruments thereon can be returned for cleaning at step 660, with the instrument data being updated to reflect the status of the instrument.

Thus, the above-described process allows instruments to be uniquely identified and consequently tracked throughout their life. This allows a history of events in which the instrument has been involved to be retrieved by scanning the barcode, determining the unique identifier, and accessing the instrument data. Whilst only limited events of cleaning, adding to a tray and use in a procedure are shown, it will be appreciated that the technique can be used to track any event. Thus each time an instrument undergoes an event, a data entry is made in a suitable database table, so if no appropriate table exists, a new one can be created. In this instance, each event instance of a respective type is provided in a suitable table row, with the rows of each table being linked to corresponding rows in the instrument data table.

This allows the instrument data to be used to retrieve details of those events in which the instrument is involved. This can be used for a number of purposes, such as identifying patients that have undergone procedures with respective instruments, or the like. Additionally, as the instrument data is linked to patient data, this also allows patient details to be used to access information regarding the instruments used on the patient.

This technique provides a number of advantages over existing tray tracking techniques that rely on an instrument being associated with a respective tray throughout their life.

For example, in such techniques, if any one instrument in the tray is unavailable, such as if the instrument it requires cleaning, repair or replacement, then this means all of the instruments in the tray cannot be used, which can in turn cause tray supply restrictions. Additionally, if instruments become incorrectly used on a tray, this prevents the history of the procedures in which the instrument is used to be determined definitively. As a result it is difficult to determine on which patients the instrument has been used.

However, in the above-described system, as each instrument is uniquely tracked, it does not matter if instruments are used on different trays in different procedures. In particular, the tray identifier is uniquely associated with the instrument each time the instrument is used. The tray identifier can then be used to determine what procedure the tray was used in at the time the instrument was assigned to the respective tray.

Thus, if an instrument is found to be used in a procedure where there is a resulting risk of contamination to other patients, or is found to be incorrectly washed, this allows the corresponding procedure and/or the cleaning batch to be identified from the instrument data. This in turn allows all other instruments used in the respective procedure, or in the respective batch to be identified and checked for contamination. Additionally, each patient that has undergone a procedure with the respective instruments can also be determined and checked to ensure that no patient contamination of infection has occurred.

### Stock Monitoring

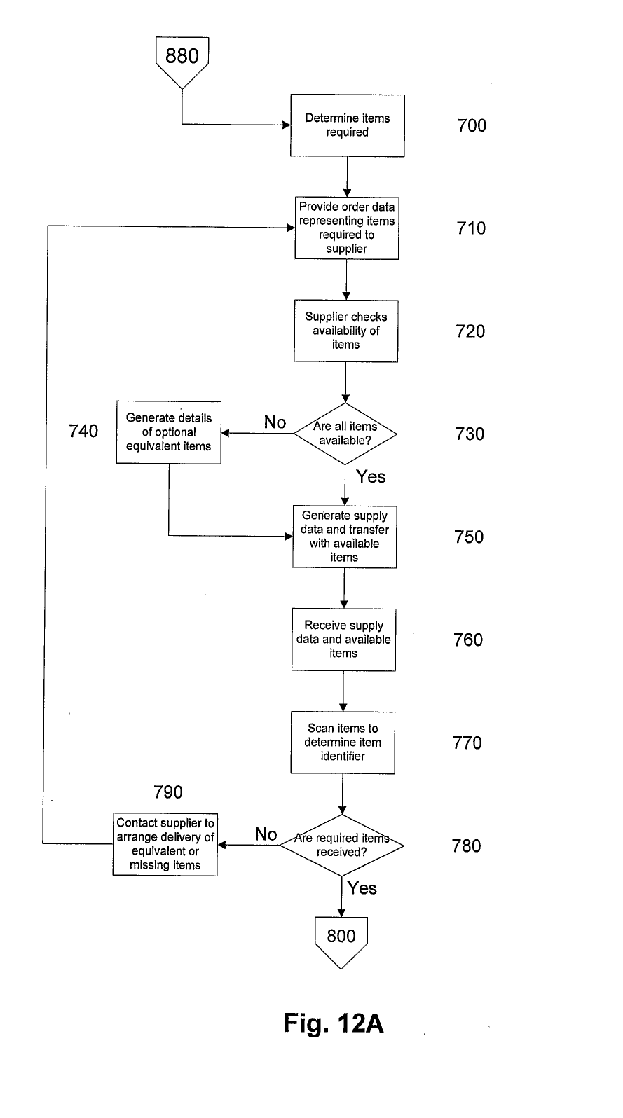

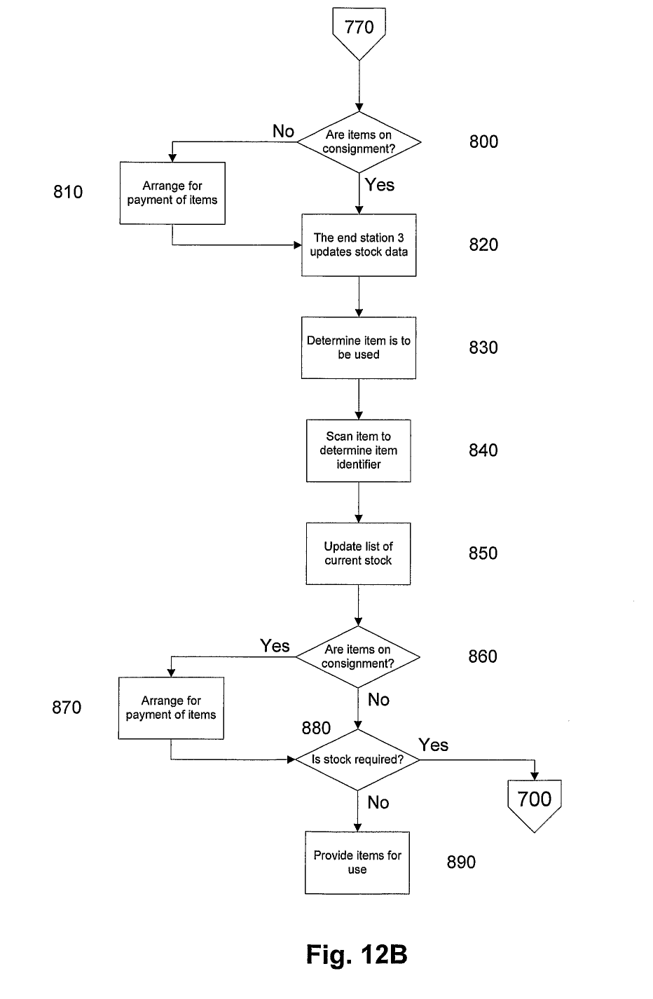

In addition to the above functionality the system described can also provide supply chain to allow ordering, payment for, and use of stock to be monitored in real time or near real time. An example of the manner in which this is achieved will now be described with reference to FIGS. 12A and 12B. This can be applied to any form of stock, such as any items, consumables, disposable instruments, other instruments, or the like, which will generically be referred to as items for clarity.

At step 700, items required are determined. This may be achieved in any one of a number of ways, and may be based for example on the needs associated with a procedure being performed on a patient. As will be described in more detail below, this can therefore involve determining a procedure to be performed on a patient, and then using preference cards or the like, to determine the items required.

In the case of a scheduled procedure, the stock is typically checked in advance to ensure that required items are ordered before the procedure is commenced. However, in the case of an emergency, items will typically be provided from existing stock, with replacement items being after the stock is used.

To check the available stock, it is typical to utilise stock data, which is indicative of items available for use, and optionally of suppliers for each respective item. The stock data is typically stored in an appropriate repository, such as the inventory management repository, which is stored centrally, for example by the base station 1, as will be described in more detail below. Thus, a user of one of the end stations 3 will access the stock data using a suitable interface, such as an inventory management module, allowing the user to check available stock and determine if further stock is to be ordered.

If it is determined that further stock is required, for example, if items are unavailable or will require replacement, then at step 710 order data representing the required items is provided to the supplier. The order data may be created in any one of a number of manners, such as by having the end station 3 create the order data using the stock data. Thus, the user can use the end station 3 to view the stock data representing live information of available stock, and where stock is running low, the user can select an associated order option.

The end station 3 then determines the preferred supplier from the stock data, if this information is available, together with any other required information, such as delivery protocols, order codes, an ETA for delivery, or the like. It will be appreciated that this information may be stored or otherwise linked to the stock data and can be based on historical data collected during previous deliveries, and/or suppliers contractual arrangements for supply.

The order data is transferred to the supplier as an electronic request, in a suitable form, such as a CDA document, secure certificates, xml document or the like.

At step 720, the supplier receives the order data, and checks to see if the required items are available for supply. This is typically a substantially automated process achieved by having the supplier implement their own supplier stock data, which can be accessed via a supplier end station 3. Thus, in this instance, the supplier end station 3 can receive the electronically transferred order data from the user end station 3, and parse this to determine the items identified therein. The items will then be compared to supplier stock data to see if the supplier has the items in stock.

If it is determined that items are not available at step 730, then the process moves on to step 740 to generate details of optional equivalent items that may be provided. Following this, or if all items are available, at step 750, the supplier generates supply data and transfers this to the medical institution that provided the order data.

The supply data includes details of the items that are available, which are also concurrently or subsequently dispatched for delivery as indicated in the supply data, together with details of any unavailable instruments. The details will typically include information regarding the items to be supplied, and can for example include one or more of:
an item description;
an item code indicative of an item type;
an item identifier, such as a barcode;
manufacturer details;
date of purchase;
warranty details or requirements;
item cost;
replacement information or requirements;
item cleaning instructions or requirements;
processing or maintenance information or requirements;
a risk level associated with the instrument;
status information, such as whether the instrument is available for use;
equivalent instruments;
item use instructions or requirements;
an indication of whether the item is provided on consignment; and,
a photo.

The supply data forwarded to the user end station 3 may also typically include all up-to-date product documentation relevant to the supplied or proposed equivalent items, such as rebate codes, catalogue numbers, descriptions, and even pictures, instructions and possibly material safety data sheets. This may also contain the invoice information for electronic invoicing.

Thus, it will be appreciated that the supplier end station 3 can automatically process received order data, determine item availability for supply, and then generate supply data. To allow the supply data to be received by the user end station 3 at the medical institution, the supply data will typically be in the form of a CDA type communication similar to the format described above.

At step 760 a user of the end station 3 receives supply data and typically uses this to update the stock data. To ensure that this is handled correctly, the user typically ensures that the items are received before the stock data is updated, although this is not essential.

Ensuring that the ordered items are correctly received can be achieved using the item identifier. Thus, in some cases, the item identifier may need to uniquely identify the item, for example, if the item is an instrument. Alternatively, however, the identifier may simply be indicative of the item type, for example, if items can be used interchangeably, or do not need to be uniquely tracked. This may occur for example, if the item is a consumable or the like.

Accordingly, in one example, such as if the item is an instrument, the item identifier may be in the form of a barcode generated and applied using the techniques described above with respect to FIGS. 8 to 10. However, in situations where the items are not required to be uniquely marked, then the item identifier may be a standard EAN product barcode, or the like.

It will also be noted that the supply data can include similar details to the instrument data described above, and as such can be used as a basis for the instrument data, thereby avoiding the need for a user to manually define the instrument data.

At step 760 the items are received and subsequently scanned, at step 770, to thereby determine the item identifier. The scanning procedure is typically achieved using a scanner that is suitable for the respective type of identifier. Thus, the scanner may be similar to the scanner 80 described above with respect to FIG. 4A to 4D, and/or may be a standard barcode scanner, depending on the respective implementation.

At step 780, the end station 3 uses the determined item identifiers to determine if all items have been received. This will be achieved by removing each scanned item from the supply data and then determining if any items remain defined in the supply data when all received items have been scanned.

If the item identifiers are not unique to each item, for example, if the item identifier is merely indicative of a specific type of item, then the supply data will typically also include a number of items ordered, thereby allowing a similar check to be performed.

In the event that certain items have not been received, for example if they are missing, or if they were unavailable, then at step 790 the process operates to contact the supplier to arrange delivery of unreceived items. This can include, for example, generating supply data corresponding to equivalent items described in the supply data, or providing an indication of missing items. Alternatively, this may include contacting an alternative supplier to source the items from a different location. The process then returns to step 710 to allow order data corresponding to unreceived items to be provided to the supplier.

This is particularly important in the case of organisations such as medical institutions, as these will typically be invoiced for all ordered items, and typically include little or no mechanism for checking if the items have been received. This is exacerbated by the fact that items may be ordered by a number of different individuals within the organisation. As a result, this can make it difficult to track which items have been ordered and consequently means that a large amount of revenue is wasted on paying for unreceived items.

For received items, the process can in any event move on to step 800, at which point, the end station 3 operates to determine whether the items are on consignment. This will typically be indicated in the supply data, although alternatively a separate of consignment items may be stored locally by the end station 3. Consignment items are generally expensive items that the organisation would be unable to pay for up front, such as prosthesis, or the like. In this case, payment for such items is only made when the item is used. However, for all other items, such as consumables, instruments, or the like, payment is generally made on receipt, and accordingly, if it is determined that an item is not on consignment, then the end station 3 will operate to arrange for payment to be made at step 810.

Payment may be achieved in a number of manners, such as by raising a purchase order and transferring this to suitable accounting entity to thereby cause the invoice to be paid. This may be achieved automatically utilising a suitable processing system, or alternatively may be performed manually as will be appreciated by a person skilled in the art. Typically however, arranging for the payment to be made will involve validation of supply data against purchase orders or requisition orders, and then validating the items against billing and invoicing requirements for automation of payments.

In any event, at step 820 the end station updates the stock data to include details of the now available items. The stock data may be formed in a number of ways. For example, in the case of instruments marked in accordance with the above described procedure, the stock data may simply include a link or other association to the instrument data. For other items, particularly if the item identifier is only indicative of the item type, the stock data usually includes details of the item together with an indication of the number of available items in stock. In this case, the item details are typically based on the details provided in the supply data, although this is not essential.

The process of steps 700 to 820 will typically be an ongoing process that is repeated as required, and is therefore used to process all incoming stock.

When it is determined that an item is required at step 830, a user will select an appropriate item from stores and then scan the item at step 840 to thereby determine the item identifier. The manner in which an item is requested will depend on the nature of the item and the respective implementation, as will be described in more detail below. At step 850 the end station 3 utilises the item identifier to update the stock data to thereby reflect that the item is to be used, and therefore no longer forms part of available stock. The manner in which this is achieved will depend on the stock data, and may therefore include reducing a count of the number of items of as certain type available, if the item is not uniquely identified, or removing the item from the stock data entirely.

At step 860 the end station further determines if the items are on consignment. In this particular instance if the items are on consignment, then the end station 3 arranges for payment of the items at step 870. This would typically be achieved using a process similar to that performed at step 810. In addition to paying the supplier, the end station 3 will also typically generate an invoice to pass the charges onto another entity, such as a health insurance company or the like. This is particularly important in the case of expensive items that are on consignment, such as prosthesis, as it is important to ensure not only that sufficient prosthesis are available for use, but also that the costs for the prostheses are recovered.

At step 880 the end station 3 may optionally determines if stock levels are low and if so contact a supplier to arrange delivery of additional items by returning to step 700. This may be achieved, for example, by comparing the number of stock items for a particular item type to a predetermined threshold and arranging to order more items in the event that the stock level is below the threshold. However, any suitable mechanism may be used.

At step 890 the item is then provided for use.

It will be appreciated that this therefore provides a mechanism for automatically tracking items in the supply chain from the supplier to their ultimate use, as well as allowing for automated payment and re-ordering to be achieved. This significantly simplifies the monitoring of stock, and allows integration with the above-described marking techniques.

It will be appreciated that as a development on the above, the system can be used to return delivered items if there is an overstock.

Additionally, the process can be used to coordinate the allocation of items to different procedures. In this case, each delivery can be associated with a booking number that associates the item as being for use by a particular surgeon and in a certain procedure. This can be achieved based on the use of preference cards, as will be described below.

The above described process can be achieved by allowing the supplier access to the repositories described in more detail below. The supplier may also implement similar applications software to that used within the medical institution, to allow stock management, and the like, albeit in a reduced form as attributes of the system such as procedure scheduling or the like may not be required.

The process can also be extended, to allow feedback of item usage to be provided to the supplier. In this example, the supplier can track received orders, thereby allowing the supplier to determine medical institutions that use certain items, to allow advertising or the like to be appropriately targeted.

Additionally, further information can be collected by, for example, storing information regarding an items intended usage at step 890. In this instance, details may be stored as item data, or the like, which can then be accessed and reviewed by suppliers. This allows suppliers to collect further information regarding the usage of items, to assist further with marketing, sales or the like. This can also extend to other entities within the medical, or other business environments.

Thus, for example, when a GP or hospital issues a prescription and then the prescription is filled at a pharmacy, the supplier can identify all the information available legally to them regarding the GP or other facility actually prescribing the product, as well as if the patient has actually used the prescription.

With appropriate configuration of data within the repository, this can be made available to the supplier without the supplier being provided with access to any patient specific (private/confidential) information. Similarly, this could be used by purchasers to find out which products are provided by which suppliers.

Searching could be achieved via a “Google” type search interface, which allows details of a product to be entered, for example in the form of product codes, item types, or the like, with the results providing information regarding product requirements, suppliers that have product, product end usage and other vital supply chain information dynamically across many suppliers and purchasers.

The presented information may be connected to or independent of patient information, for example, depending on the circumstances in which the information is provided. Thus for example, medical personnel may be required to undergo authentication to allow patient information to be reviewed. This can therefore be used as both a powerful business and possibly public safety tool, for example to allow the government to determine the extent of dissemination of a certain drug, over a certain time period and/or in a certain demographic area, alerting the government to an outbreak, or the like.

### Item Requirement Determination

In the example processes discussed above, such as at step 700, it is necessary to determine if an item or instrument is required for use. The manner in which this is achieved will depend on the type of instrument or item being used, and the nature of the corresponding event.

In the case of events, such as cleaning, an indication that items are required may be generated automatically by the end station 3 based on the batch data. Thus, the end station 3 can use the stock data to determine when an item was last provided, and also use the batch data to determine the number of specific types of cleaning events that have occurred since that time. This, in turn allows the end station 3 to calculate when a replacement item is required. Thus, for example, if the item is a consumable such as cleaning fluid, the batch data can indicate a typically number of cleaning events that can be performed before the fluid is used up. In this case, when the end station 3 generates the batch data at step 410, the end station 3 will check when the item was last replaced, and in the event that this exceeds a predetermined number of cleaning events, additional cleaning fluid will be requested from stores.

Alternatively, the requests may be provided manually. Thus, for example, cleaning fluid may be wasted during the cleaning process, for example, if some of the fluid is spilled. Accordingly, replacement cleaning fluid may be required before the predetermined number of cleaning events have occurred, in which case a user of the end station 3 can submit a request to stores manually via an appropriate mechanism.

In the case of items used in a medical procedure, the items used will generally depend not only on the procedure involved, but also on the preferences of the medical personnel involved in the procedure. For example, each surgeon may use a different combination of instruments for performing the same procedure.

To account for this, the system is adapted to store preference card data. The preference card data indicates, for each medical practitioner, and for each type of procedure they are to perform (which is typically determined based on an association to the procedure data), the instruments and other items required. In this case, when a procedure is scheduled, and the practitioner involved determined, a user of the end station 3 can access the preference card, which indicates each of the items and instruments required. This information is then used at step 700, to ensure the correct items are made available.

It will be appreciated from the above, that when the preference card is used to obtain items for use in the procedure, this can be used to automatically trigger the ordering of replacement items.

### Patient Information Collection

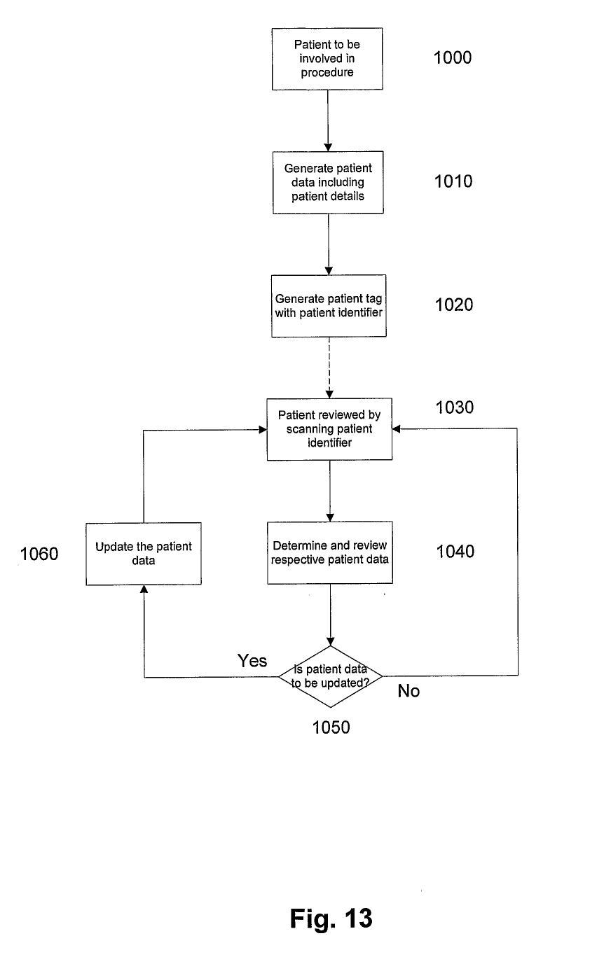

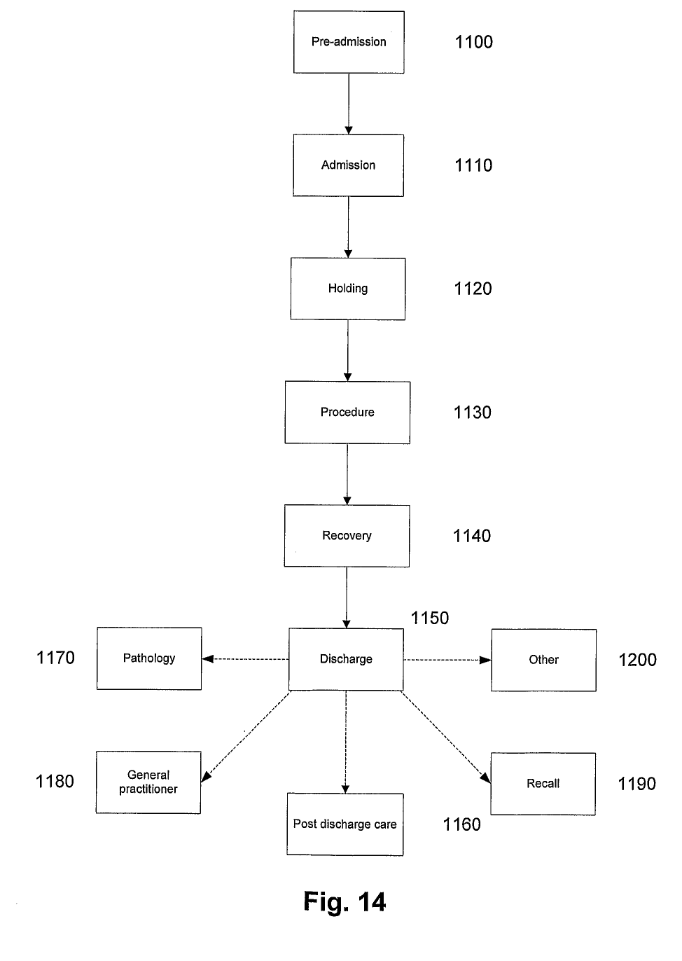

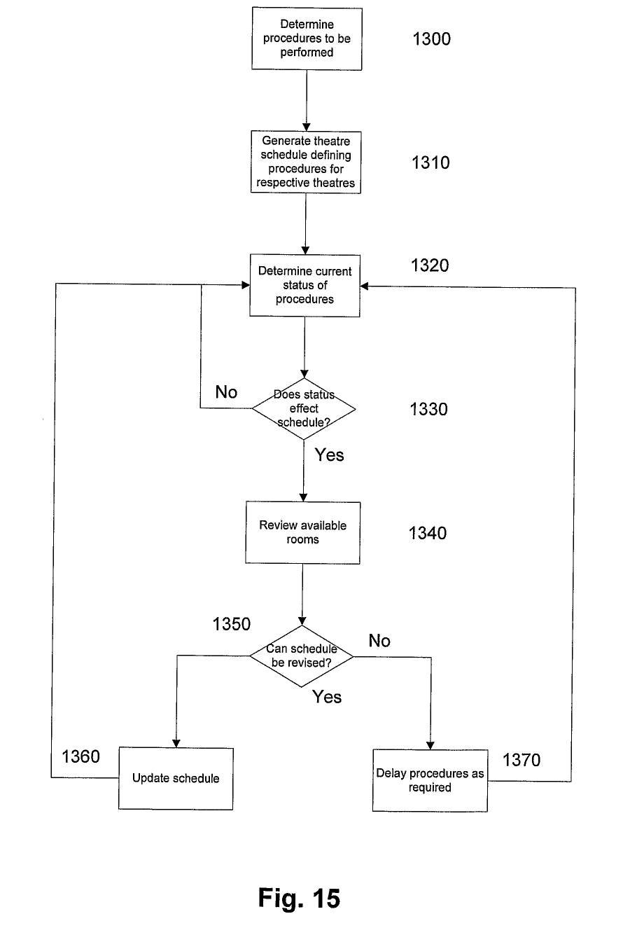

In addition to the above-described processes, the current system can also operate to collect and collate information regarding a patient and their progress through a procedure, as will now be described with reference to FIGS. 13 and 14. As will be described in more detail below, this allows information regarding use of assets or stock, such as items and instruments, to be associated with the patient, thereby allowing subsequent identification of assets, such as instruments, used on the patient.

The manner in which data collection occurs will first be described with reference to FIG. 13. At step 1000 the patient is to be involved in the procedure. Accordingly, the end station 3 is used to generate patient data including patient details at step 1010. The patient details will include information allowing the patient to be identified, as well as any information relevant to the procedure, and may therefore typically include information such as:
name;
address;
phone number;
other contact details;
details of prior medical conditions;
details of allergies;
current medication taken;
a current patient status, including for example:
   blood pressure;
   temperature;
   location;
   procedure stage; and,
other relevant information.

As an alternative to generating patient data, patient data may be received from another source. Thus, for example, if the patient is referred from another medical institution, the patient data may be received from multiple other sources, such as one or more other medical institutions and/or other internal facility systems, thereby avoiding the need to create patient data already in existence.

In any event, the end station 3 will generate a patient tag associated with a corresponding patient identifier. The patient tag is typically in the form of a wristband or other wearable device that can be fixed to the patient to allow the patient to be identified by medical personnel. In one example, the patient identifier is a linear barcode, such as a barcode defined in accordance with EAN protocols or the like. In any event, irrespective of the nature of the patient identifier, the patient identifier is used to uniquely identify the patient within the medical institution, and therefore is unique for the respective institution. The patient identifier is associated with the patient data, allowing it to be retrieved by the end station 3.

Thus, at step 1030, when a stage in the procedure is to be performed, or when the status of the patient is updated or checked, the patient tag is scanned using a suitable scanner, such as a barcode scanner, with details of the scanned identifier being transferred to the end station 3. At step 1040, the end station 3 will determine the access the corresponding patient data and display this, thereby allowing the patient details to be reviewed by medical personnel.

At step 1050 it is assessed if the patient data needs to be updated and if so the process moves on to step 1060, allowing the medical personnel to update the patient data using the end station 3. Once this is completed or in the event that no update is required, the returns to step 1030 allowing the patient data to be further reviewed as required.

Thus, it will be appreciated that utilisation of the barcode associated with the patient allows the patient tag to be scanned and the corresponding patient data displayed on a suitable end station 3. In the event that an end station 3 is a portable device, such as a PDA, Tablet PC, Lap-top or the like, this allows medical personnel to move around the medical institution and view and update information regarding patients as required.

In this particular instance each time details of the patient are measured, such as blood pressure, temperature or the like this can be added to the patient data together with a corresponding time indication so that a complete record of the patient's history is displayed.

An example of the circumstances when this may be used is shown in FIG. 14, which shows the general stages involved in performing a procedure on a patient.

Thus, as shown at step 1100 the patient undergoes pre-admission. Such a pre-admission stage is generally used to prepare the patient for the operation by allowing tests, such as blood pressure checks, or the like to be performed, to allow the patient data to be generated, as well as to ensure the patient does not eat or drink prior to surgery or the like being performed. Whilst the pre-admission may be performed in a hospital, it is also possible for the pre-admission process to be undergone at a different environment, such as a hotel or the like.

At step 1110 the patient is admitted to hospital during an admission stage. At this point, it is typical for the patient details to be checked, and the patient assigned to a bed or the like. Following this, and once the procedure is ready to be performed, the patient is transferred to holding area at step 1120 to allow the patient to await surgery. At this point, the patients identifier will typically be scanned and the patient data compared to a schedule indicative of the procedure to be performed, to thereby ensure that the correct patient is provided for the procedure.

At step 1130 the procedure is performed. During this process, the patient data may again be checked and updated as required, for example to allow the status of the patient during the procedure to be recorded. This also allows details of the procedure to be recorded, although alternatively this can be achieved by generating procedure data, which is then associated with the patient data, for example by utilising suitable linking within a database.

At step 1140, the patient enters the recovery stage with details of the patient's recovery been recorded in the patient data, before the patient is discharged at step 1150. Thus, it will be appreciated from the above, that this provides a mechanism for maintaining a detailed record of the patient, and in particular, their health status at each stage during a procedure. This can include, for example, details of health status indicators, such as temperature, blood pressure, or the like.

Thus, this technique can provide an audit trail of the patient's health status, with the results of each monitoring step being recorded together with information such as the time and date, to allow the progress of the patient through the procedure to be subsequently reviewed.

It will be appreciated that the information collected during this process may be used in any one of a number of environments, such as post discharge care 1160, pathology 1170, by a general practitioner or other medical personnel at 1180, in recall of patients, equipment or the like at step 1190, and for a number of other uses at step 1200.

### Procedure Scheduling

In order to allow a number of procedures to be performed in a number of different theatres, or other appropriate rooms, it is necessary to schedule the procedures and the use of the rooms. This is a complex task particularly when it must be taken into account that procedures can overrun if complications occur. Accordingly, the above described system can also be used to maintain and update a theatre schedule which sets out details of the times and locations in which respective procedures are to be performed.

In order to achieve this at step 1300 it is necessary to determine procedures to be performed. This will typically be in the form of a list of procedures, together with additional information such as the preferred ordering, or relative importance of the procedures, together with an indication of additional requirements, such as requirements for the room facilities or the like. This information may be derived from requirements data as will be described in more detail below.

At step 1310, the end station 3 generates a theatre schedule. In this instance, the end station 3 will utilise a scheduling algorithm to assign procedures from the list to respective ones of a number of different available operating theatres. This will take into account requirements for the procedure, thereby ensuring the theatre to which the procedure is assigned is suitable for such use. This will typically utilise generic scheduling algorithms, or may be performed manually, depending on the preferred implementation, and this will not therefore be described in any further detail.

Once the schedule is defined, it is important to ensure that this is maintained in as an efficient manner as possible to allow the maximum number of procedures to be performed.

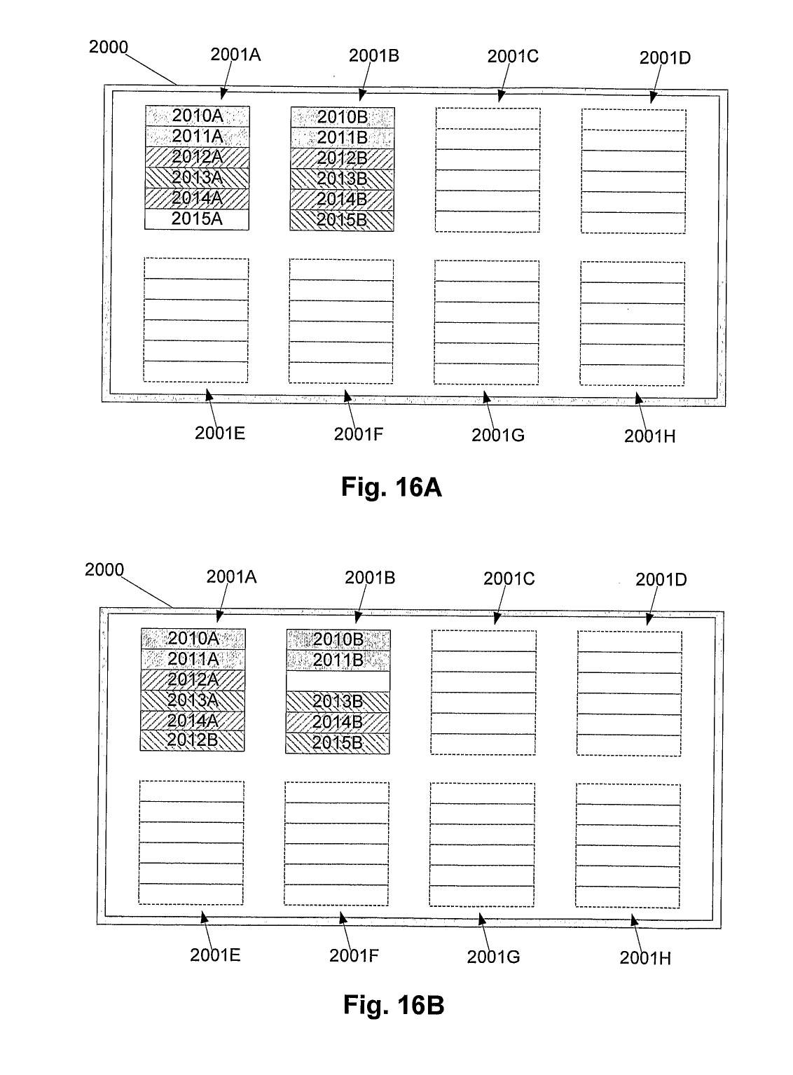

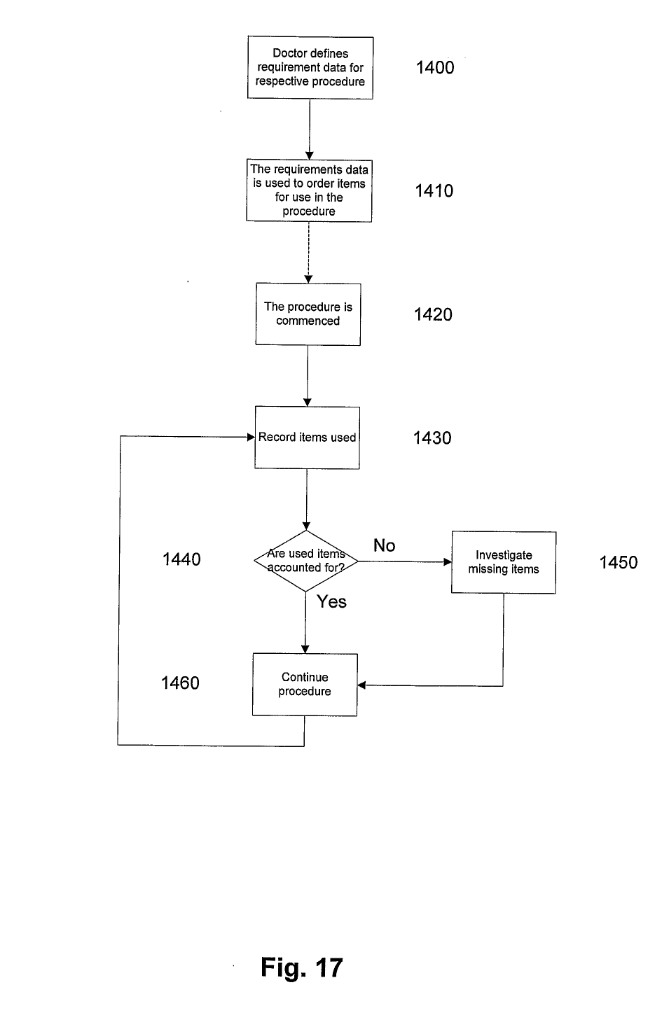

Accordingly, at step 1320 the end station 3 is used to determine the current status of procedures, using this to generate a GUI showing the current procedure status. An example of a suitable GUI is shown in FIG. 16.

As shown the GUI 2000 includes a list 2001 associated with each of the available rooms, as shown at 2001A, . . . 2001H. In this example, and for the purpose of clarity, the explanation will focus on the use of two theatres only, represented by the lists 2001A, 2001B, although it will be appreciated that the techniques are equally applicable to any number of theatres.

In any event, in this example, each of theatres utilises six time slots, shown generally at 2010, 2011, 2012, 2013, 2014, 2015. Each slot is therefore typically of a predetermined time length, such as one hour, or the like.

In use, each time slot is used to represent whether a procedure is scheduled to performed within the theatre during the respective time slot. This allows, an operator is presented with the GUI and can therefore view the procedures scheduled for different theatres at different times. When the end station 3 presents the GUI the end station 3 utilises the current status of procedures to generate an appropriate status indication on the schedule. The status indication is based on a comparison of the current status of the procedure with a predicted status.

Thus, for example if the procedure is running behind time the current time slot for the corresponding theatre 2000 will be highlighted in a different colour, such as red, whereas if the procedure is running on time a green status indicator colour will be provided. Scheduled procedures not yet in progress are then indicated in a further colour.

In this example, the time slots indicate the status as follows:
time slots 2010A, 2011A—procedure in progress and on schedule;
time slots 2012A, 2013A, 2014A, 2012B, 2013B, 2014B, 2015B—procedure scheduled to be performed;
time slots 2010B, 2011B—procedure in progress and behind schedule;
time slots 2015A—no procedure scheduled;

Accordingly, an operator will examine the available theatre rooms and in the event that any one of the procedures is behind schedule can operate to update the schedules.

Thus, at step 1320 if the operator determines that the current status does not effect the schedule the process will return to 1310, allowing the GUI 2000 to be updated as the procedures progress, as required.

However, if the operator determines that the current status will effect the schedule, for example if a procedure is likely to overrun, then the operator will determine other available rooms at 1330.

Thus in this example, the procedure being formed in the theatre 2000B is currently overrunning, and accordingly, the operator must make additional time available, by moving one of the procedures subsequently scheduled for the respective operating theatre 2001B to another room. Accordingly, the operator can determine if the schedule can be revised at step 1340 by determining if the procedure can be moved to a different theatre.

In this example, the theatre 20021A includes an available time slot, and accordingly, this is achieved by moving the procedure scheduled for the time slot 2012B, to the time slot 2015A, as shown in FIG. 16B. This is achieved by having the user drag and drop the procedure from the representation 2001B to the representation 2001A.

It will be appreciated that the manner in which the operator reschedules procedures will depend on a number of factors, such as the relative importance of the procedure. Thus, for example, if the procedure at 2012B is urgent, each of the procedures at 2012A, 2013A, 2014A may be delayed to allow the procedure shown at 2012B to be performed at the originally allotted time, albeit in a different theatre.

To assist with this, the GUI will typically allow the operator to view details of the scheduled procedures, for example, by allowing the operator to click on one of the time slots and view procedure details in a separate window or dialogue box.

Additionally, the GUI can include a list of as yet unscheduled procedures. This allows the operator to add procedures to the schedule, and it will be appreciated that this technique can be used to define the initial schedule at step 1310.

In any event, when the GUI is manipulated, the end station 3 will update the schedule based on the revisions made by the operator and then return to step 1310 to allow monitoring to continue.

Alternatively if it is determined that the schedule cannot be updated, for example if no other rooms are available, then the user can simply delay a procedure by cancelling it as required. At this point, the procedure will be returned to the list of as yet unscheduled procedures, allowing the procedure to be reallocated to a theatre as time slots become available.

### Electronic Count Sheet

During procedures, it is important that all medical items and instruments are accounted for. In particular, this is important to ensure that instruments and/or items such as swabs or the like, are not left within the patient. This is typically achieved using a count sheet, and an example of utilising the above described system for providing an electronic count sheet will now be described with respect to FIG. 16.

In this example, at step 1400 a doctor or other medical personnel defines requirements data for a respective procedure.

The requirements data will be generated on a case by case basis as each doctor will generally have respective requirements for each procedure. In any event, the requirements data typically includes a list of the items or instruments used during the procedure, and can therefore be formed from the preference card data discussed above. Accordingly, the requirements data can be used to order items for use in the procedure, at step 1410, as has previously been described.

At step 1420 the procedure is commenced, with any items used being recorded at step 1430. Periodically at step 1440 medical personnel will determine if used items are accounted for. If not the process can move on to step 1450 to allow the missing items to be investigated. Otherwise the procedure can be continued by confirming the items are accounted for and then returning to step 1430 to allow use of additional items to be recorded.

It will be appreciated that this provides a mechanism for continually updating a list of used items with this being periodically checked to ensure the used items are accounted for and not left within the patient or the like.

To achieve this, the medical personnel may be presented with a GUI by an end station 3 during the procedure, with the GUI displaying a list of each item available for use. As each item is used, it is selected on the list, with the list being modified to indicate the item has been used. When an item has been accounted for, the item can be selected a second time, allowing its status to be further updated to reflect that it has been used.

### Data Management

It will be appreciated that the techniques described above can be combined to provide for an integrated hospital management system that allows tracking of item use, procedure scheduling, patient monitoring and stock monitoring.

In one example, this is achieved by using an association between patient data indicative of a patient involved in a medical procedure and asset indicating data indicative of assets used within the medical environment. This allows information regarding assets associated with the patient during the performance of the procedure, to be subsequently retrieved and reviewed. Additionally or alternatively, this allows inventory control and ordering, billing and the like to be achieved. The indicating data can take any one of a number of forms such as instrument data regarding instruments used, as well as inventory data, stock data or asset data regarding the assets used.

To achieve this, the system utilises a number of repositories for storing data including:
an asset repository;
an instrument repository;
corporate data repository; and,
inventory management repository.

### Asset Repository

The asset repository is used to store asset data, which provides details of any asset used within the medical institution, including all assets, such as medical instruments, items, consumables, other stock, or the like. Entries in the asset repository are provided so that they contain information that is common to each instance of the particular asset, thereby allowing details of an asset, to be provided by referencing the asset repository. This can include information such as an asset product code, the manufacturer/supplier of the asset, the manner in which the asset should be used, maintenance information, cost, or the like.

### Instrument Repository

The instrument repository is used to store details of each instance of a physical instrument within the medical institution, such as the instrument data outlined above. This is achieved by providing information that is specific to the respective instrument instance, and then linking this to an appropriate entry in the asset repository. Thus, the asset repository will include information that is common to each instrument of a specific type, with each physical instance of an instrument within the medical institution being identified by an entry within the instrument repository.

When a user is required provide instrument details for a new instrument at step 290 above, the user can simply access the asset repository and determine if details of that type of instrument already exist. If so, the instrument details are provided in the instrument repository by providing specific information such as the date of purchase of the instrument, and the associated unique identifier, and then linking this information to the appropriate entry in the asset repository.

So, for instance, if the user is entering details of a scalpel that has just been assigned a unique identifier, the user can achieve this by accessing the asset repository to determine if a record for a scalpel asset of the same type exists. In this case, the user can then simply create a new scalpel instance in the instrument repository, which includes the unique identifier and therefore distinguishes the scalpel from each other scalpel, and then link this to the scalpel asset entry in the asset repository, to thereby provide the necessary details of the instrument. If no suitable entry is present in the asset repository, then this will need to be created.

### Inventory Management Repository

This is used to store information, such as stock data, regarding the inventory within the medical institution, as described in the stock monitoring procedure above with respect to FIGS. 12A and 12B.

Thus, stock data is typically formed by entries in the inventory management repository, which reflect numbers of specific asset instances, such as items, and consumables in stock within the medical institution.

Thus, entries within the inventory management database reflect specific item instances, with a link being provided to information common to the each item of a specific type being provided in the asset repository. Thus, for example, bandages of a given type may have a description and product code, which is stored in the asset repository, with each bandage within the medical institution being identified by a unique entry in the inventory management repository, allowing current stock levels to be easily determined.

### Corporate Data Repository

This is used to facilitate the management and/or compilation of data for patient related and/or influenced activities. This allows integration between existing systems and the above described processes and their corresponding implementing applications, as well as to provide data management pathways.

To achieve this, the corporate data repository stores, or provides references to data which forms the patient data, electronic count sheet data, preference card data, or the like.

Thus, the data collected by systems utilised in the above described processes, as well as other systems within the medical institutions may either be stored in the corporate data repository or in the case where the data is stored in other databases, simply addressed by the corporate data repository. This then enables the applications to manage this data for process management and/or control/manipulation and then ultimately updating other systems with the data compilation.

The corporate data repository is generally built from HL7 messaging or ODBC connection to such other databases or information sources, could be from triggering an event that in turn provides a simple XML string for the corporate data repository to interact/be populated with.

An example of the databases connected to and information managed by the corporate data repository are as follows
Purchasing
Sterilization department processing
Maintenance
Accounting
Asset repository
Pharmacy
Theatre list or theatre management systems
Billing
PAS (Patient Administration System)

Accordingly, data such as patent data can be formed from a combination of patient information held in the corporate data repository and/or information created in specific applications and/or stored in specific databases used for implementing the above described processes.

The patient data is appropriately linked to each of the other repositories to reflect interactions with the patient. This is performed in advance of procedures being performed on the patient, thereby allowing inventory for the procedure to be ordered and prepared. Additionally, once the procedure has been completed, this provides an effective audit trail of interaction with the patient, allowing identification of procedures performed on the patient, and the respective assets used during this process.

This makes the data model patient centric, so that once a patient has been identified, all information relevant to patient may be retrieved.

Medical staff preference cards, electronic count sheets and scheduling information may also be formed from a combination of data from the corporate data repository, asset, instrument and inventory repositories and information created in specific applications, and/or stored in specific local or centralised databases.

### Data Collection

In addition to manual creation of the entries in the repositories, data may also be collected automatically from existing hospital systems. This can include for example, the following systems:
Purchasing
Sterilization department processing
Maintenance
Accounting
Asset repository
Pharmacy
Theatre list or theatre management systems
Billing
PAS (Patient Administration System)

The collected information will relate not only to surgical instruments but all inventory, from a consumable, fixed asset used or replaced, consignment asset, drug allocations, pathology requests and results, blood types and usages, loan items or impress asset of actions taken by a clinician or healthcare worker enhancing the data set for these other systems. This information can even be expanded to bed or chart locations, remotely accessed by specialists and GPs.

### Data Access

To provide for centralised access to the data by different medical institutions, the data is typically stored in databases hosted by a central server or base station, such as the base station 1 shown in FIG. 5. This allows the repositories to be made available to medical institutions via a web based, or another similar portal, using for example, the end stations 3. Alternatively, this can be dynamically presented e.g. to a GP via a connection integrated to the GP practice management solution for viewing and updating the core system with such information as patient related and/or inventory management such as drug usage or referrals requirements or patient historical clinical information.

The repositories can be implemented and accessed at any one of a number of different levels, such as
GP (general practitioner)
Hospital
Area health service or hub
State or nation level

Thus, for example, a separate base station 1 may be implemented at each of these levels, so effectively separate repositories are provided. In the case, for example, of an area health service or hub, the base station 1 will therefore collect data from each of the medical institutions, such as hospitals, GPs, or the like, and store this centrally. Each medical institution within the area is then able to access the information via an end station 3 provided within (and/or externally to) the institution. As mentioned above, this can be achieved via a patient centric model, so this can be performed on the basis of patient details alone.

Thus, for example, if a patient is received within the emergency department of a hospital, the hospital can use the patient's details to access the repositories and determine the patient's medical history. This includes details of previous procedures performed on the patient within the area health service or remote health service presenting as a hub or state level data repositories as in the above, even if these are performed by other medical institutions. Furthermore, this includes details not only of the procedures themselves, but also of the assets involved in the procedure, such as the medical staff, the instruments used, drugs and the like. This allows a rapid check to be made to determine if any of these assets could have had an impact on the patient's current health status.

Accordingly, the corporate data repository, asset repository, inventory and instrument repositories can all be accessed dynamically from suitable system, such as suitable applications software implemented on a processing system, or via other projects, databases or the like. This means that any above described process and/or process management solution can access directly, or from a local source database, information required to manage such processes, as well as to retrieve such data for its own use and/or that of the other systems from the core system.

It will be appreciated that the entity administering the repositories may administer the identifiers used in marking instruments, but this is not essential and entirely separate systems may be used. The reference to the base station 1 is therefore for the purpose of example only, and particularly to illustrate a suitable apparatus configuration, and is not therefore intended to be limiting.

### SPECIFIC EXAMPLE

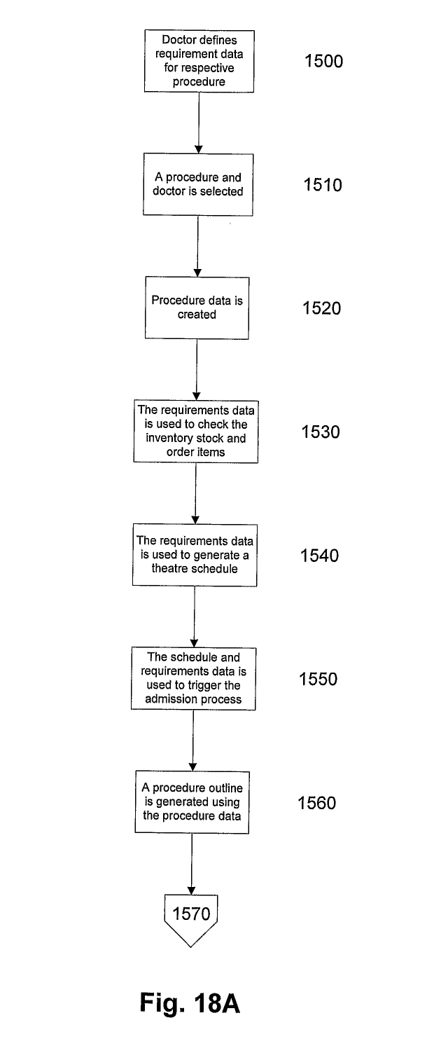

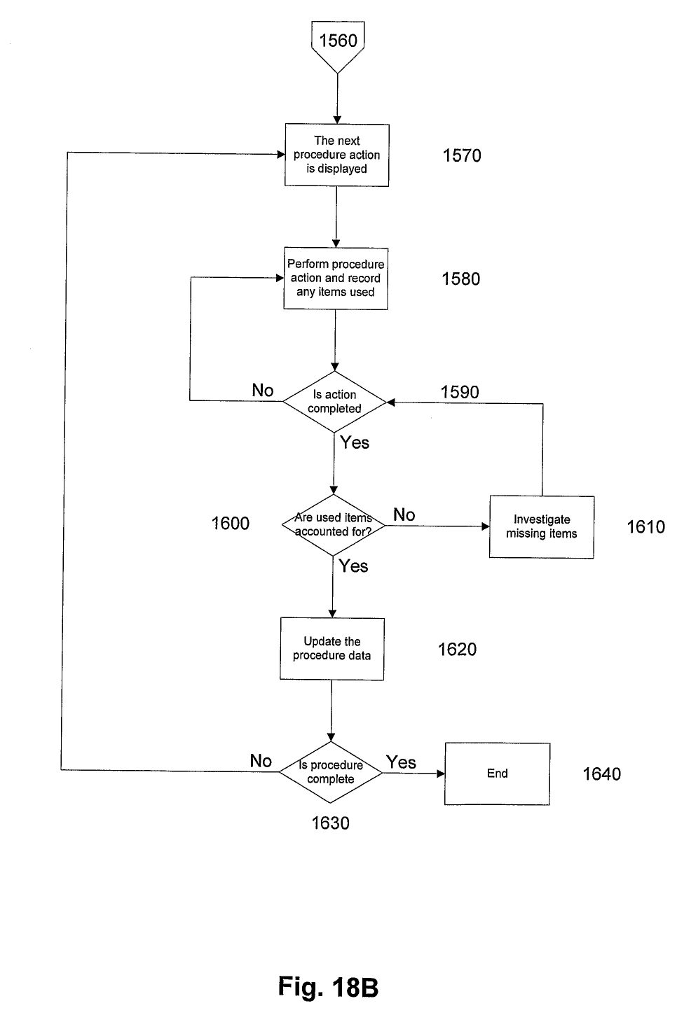

A specific example of the manner in which the above described processes may be integrated for the purpose of performing a procedure on a patient will now be described with respect to FIGS. 18A and 18B.

At step 1500 the doctor defines requirements data for a respective procedure. This will be a one of process that is performed each time the doctor defines a new procedure, and is not performed for each patient is admitted. This information may be stored either in a central repository, or locally within a medical institutes own database system, and is typically based on, or formed from any preference cards, procedure data, or the like, that has previously been defined. The requirements data will typically include information such as the items and instruments required to perform the process, and will therefore typically include links back to the asset, inventory, instrument repositories and billing systems.

Additional information may also be included, such as:
an estimated procedure duration;
theatre requirements;
medical personnel required for the procedure;
the actions involved in the procedure;
items required for the procedure;
instruments required for the procedure;
restrictions on patient health status indicators;
admission procedures; and,
other relevant information
Procedure costs

At step 1510, once a patient has been diagnosed, a procedure and a doctor for performing the procedure are selected. This may be done in any manner and will depend on whether the patient has been referred, is admitted as an emergency, or the like.

At step 1520 procedure data is created. As described previously, the procedure data is data corresponding to details of the respective procedure to be performed on the respective patient and is therefore unique to each procedure/patient combination. The procedure data allow a concordance between the patient and details of the procedure performed to be subsequently determined. In simple terms this may be no more than an association between appropriate the patient data described above in FIG. 13 and the requirements data for the associated procedure. The patient data may be generated either by accessing a suitable repository, by receiving details from a referrer, or by interviewing the patient, depending on the particular circumstances.

In any event, once this has been created, it is possible to subsequently access the requirements data and confirm what procedure is to be performed, and what assets are required. This can be used for the purpose of subsequently tracking asset usage, as well as billing, scheduling or the like.

Accordingly, at step 1530 the requirements data is used to access the inventory repository and check whether sufficient inventory is available for performing the procedure. This will also trigger the ordering of replacement inventory, such as disposable instruments, consumables, or other items that are to be used in the procedure. This ensures that minimum stock levels are maintained at all times and increase income via accurate automated billing or items used.

At step 1540, the requirements data is used to schedule a time at which the procedure can be performed. This will involve determining when an appropriate theatre is available, for example based on the scheduling process described above. This may also be based on other information, such as an assessment of the required urgency of the procedure, which may be assessed by other medical personnel, for example, as part of a referral process.

The end station 3 will then monitor the schedule and then use the requirements data to trigger the admission process at step 1550. Thus, the end station 3 will monitor a schedule and at a predetermined time before the procedure is scheduled to be performed, operate to arrange for the patient to undergo the steps of pre-admission, admission and holding, as described in FIG. 13 above.

At step 1560 a procedure outline is generated using the procedure data. The procedure outline includes a list of actions that will be performed during the procedure together with the corresponding items used for that action. At step 1570, when the procedure is being performed, the next procedure action is displayed on an end station 3 provided in the corresponding theatre.

At step 1580 as the corresponding actions are performed, any items used will be recorded by having one of the medical personnel enter information utilising a suitable GUI. Thus in general, the action displayed will include a list of typical items used. In this instance as an item is used by the doctor or other medical personnel an operator can simply select this on the action list for example by touching a suitable touch screen or the like.

It is determined that if the action is completed at step 1590 and if not the process returns to 1580 to allow any additional items used to be recorded. Once the action is completed at step 1600, it is determined if all used items are accounted for. This is achieved for example by having the medical personnel review the list of items and ensure that each one of the items is either disposed of, still available for use, or used.

If any items are determined to be missing at step 1600 these are investigated at 1610 with the process returning to 1590 if further action is required, for example to recover the item from the patient. Once all used items are accounted for the procedure moves on to step 1620 to update the procedure data.

This will typically be achieved by having one of the medical personnel select an action option on the GUI which indicates the action is completed, which will in turn automatically update the procedure data. This typically involves updating the procedure data with instrument data reflecting the actual instruments used during the procedure, as set above for example at step 650. Thus, an association is created between the patient and the instruments, so the instruments used on the patient can be subsequently determined. In one example, whilst the use of procedure data is described, this could be as simple as creating an association, such as appropriate links between the patient data and the entries for the respective instruments in the instrument repository,

Once completed, the process moves on to step 1630 to determine if the procedure is complete. If not the process moves back to step 1570 allowing the next procedure action to be displayed and this process is then repeated until the procedure is completed at step 1640.

It will be appreciated from this that the procedure data for a respective procedure is updated in real time, allowing this to be used in scheduling other procedures at step 1320.

Furthermore, at the end of the process, the procedure data, which as mentioned above may be no more than a mapping between the patient data, core systems and entries in other ones of the repositories, will reflect what the items and specific instruments used in the procedure, allowing the information to be subsequently retrieved based on patient information.

### Usage

Accordingly, the process provides a physical control system that operates to scan and validate items used in medical institutions. This information, together with details of procedures, item and instrument usage are then stored against patient information allowing the system to provide control management based on the patient information.

This allows a range of different functions to be performed, including those outlined above, and including:
Purchasing—updating of asset levels
Sterilization department processing—validation of items for process and patient safety
Maintenance—allocation of use and maintenance cost—whole of life cost—purchasing
requirements—business requirements
Accounting—business intelligence reporting on billable items, rebateable items, asset usage against costs based upon a patient episode—other KPI and business reporting and modelling
Asset handling—ensuring the accounting and purchasing and other systems in the facility use and recognise the same base item information across all systems even if the users call or identify the item under a different description.

Pharmacy—allocation, stock control and usage—combine with patient and procedure giving a total consolidated view of the cost, time and materials against a patient centric view.

In addition to this, patient information can be retrieved from the system and returned to internal systems within the medical institution, for use in, for example:
Theatre list or theatre management systems,

Billing—allocation of stock against the patient for billing against a patient episode—increased revenue by accurate management of cost and items used.

PAS (Patient Administration System)—updating of patient information if more current from other systems.

This service can update, view and pass information about a patient from the data repositories to the different levels for enabling these systems.

Such an example would be a GP wants to know what drugs have been used on a patient and therefore can see this or add this info on their own patient system—add the new drugs they prescribe and then update the repository and or keep the information in their own GP system.

Or an area health service may wish to use the system for central purchasing solutions, or national cost reporting and/or the same for any level, where the requirement is for a consolidated view from an intelligent system that allows either the storage of or knowledge of where the data is and which core systems require it, to then supply this information as managed to these systems for data processing based on an action of the user to update the repository either with the new data or a reference of what systems hold what data based upon a patient centric data repository module.

Thus, this allows for an audit trail to be provided relating to procedures a patient has previously undergone, or is to undergo, together with details of all consumable items and the like used therein. This not only allows this information to be subsequently retrieved and reviewed, but also allows the information to be used in ancillary services, such as billing the patient for the procedures and items used, as well as ordering replacement stock.

### Further Features

A number of variations and additional features of the above described process will now be described.

The bonding material can be applied to the instrument surface using any one of a number of techniques. This can include for example manually painting the bonding material onto the instrument surface, or manually applying an adhesive dot of material. However, as an alternative a suitable printing system can be integrated into the laser system 70.

In this instance, when the laser system 70 is to be used to expose the bonding material, the end station 3 first controls the laser system 70 to selectively position a print head over the aperture 53. The print head is used to print bonding material onto the instrument surface thereby ensuring that the bonding material is provided aligned with the aperture 53. The laser system 70 can then be moved or otherwise controlled to allow the laser beam to expose the bonding material.

Similarly, it is possible for the laser system 70 to incorporate a suitable detection system, such as a scanner 80, to allow the barcode provided on the instrument to be detected. This allows the laser system 70 to automatically scan the encoded instruments to allow checking of the clarity of the barcode to be performed. This obviates the need for an operator to manually handle the instruments to perform the scanning and subsequent check of the instruments.

It will be appreciated that in the event that the laser system 70 includes an imaging system to allow detection of the position of the aperture 53, or the bonding material, that this can also be used to provide appropriate scanning of the barcode to allow barcode clarity to be checked.

The trays can be identified in the same way as the instruments utilising an appropriate barcode which encodes a unique identifier corresponding to the tray identifier. This allows trays to be uniquely tracked by scanning the barcode in a manner similar to that described above with respect to the instruments.

The system also allows costing information to be determined. In this instance, a cost can be defined for each of the events performed. The cost can either be predefined, for example, with a set cost for each event, or may alternatively be defined as part of the event data, each time the event is performed. This allows a cost total to be determined for various events, as well as for each instrument.

Additionally, the process can be used to monitor stock levels, for example, by tracking the number of instruments in circulation, thereby allowing replacement instruments to be automatically ordered when required.

The use of appropriate rules in monitoring the number of events occurring can be used in order consumables used in different events, such as cleaning materials, as well as monitoring maintenance of machines used in cleaning.

As mentioned above, appropriate business rules can be used to restrict the use of instruments, thereby ensuring instruments are only used is procedures falling into appropriate risk categories, as well as to ensure instruments of different risk levels are not used together.

The system can also be used to ensure that correct maintenance is performed on instruments, for example, by monitoring the number of times the instrument has been used, and generating an alert if scheduled maintenance is due.

Whilst the above process has been described with respect to an end station 3, it will be appreciated that medical institutions, such as hospitals, general practitioners, private clinics, and the like will typically use a number of different end stations 3, depending on the implementation. Typically, this is achieved by having each of the end stations 3 access a central repository which stores the instrument and other event data, typically via a suitable LAN, WAN, or the like. Thus a single database may be provided for all of the instruments used within an organisation. Access can be via wired or wireless connections, and it will therefore be appreciated that the end stations may be formed from portable devices which include a suitable scanner 80, thereby allowing instruments to be scanned, and the corresponding instrument data displayed. Interaction with the instrument data can then be performed as required.

By having access to the data restricted in an appropriate manner, this allows any authorised user of the system to access the instrument data. Additionally, the actions that can be performed with both the data and the instruments may be associated with respective authorisation levels. Thus a user with only limited access rights may be able to view data but not modify the data n any way. In this case, when the user scans the instrument, an alert can be generated indicating to the device user that they are unauthorised to perform certain actions with the instrument or associated data.

In the above described examples, when instruments and/or trays are provided for use in procedures, it is typically for the instruments and/or tray to be provided in a sterilised package that is only opened upon use in the procedure. In this instance, it is difficult to scan and correctly identify instruments through the packaging. Accordingly, it is typical to include an identifier on the packaging.

The identifier used on the packaging may be similar in nature to the instrument identifier, and in the case of a single instrument being wrapped, could be the same as the identifier of the packaged instrument. However, typically it is far easier to encode information on, and read information from the packaging, due to the inherent different properties of packaging material compared to the instruments. Accordingly, it is typical to use a standard barcode, such as an EAN barcode.

In this instance, the identifier provided on the packaging is uniquely associated with each instrument contained therein, with subsequent tracking of the tray and/or the instruments being achieved by tracking the barcode associated with the packaging.

Thus, for example, when a tray is received for use in a procedure, the tray packaging is scanned and the barcode determined. The end station 3 can then access the instrument data for each instrument provided in the packaging, allowing the instrument data to be associated with the corresponding procedure data as previously described.

The term instrument is not intended to be limiting and the techniques could be applied to any item used in medical procedures. The terms item and instrument have therefore been used only for clarity purposes, and the processes involved could be applied equally to instruments or items as appropriate.

Additionally, whilst the above described techniques are focussed on the treatment of patients in medical environments, such as medical institutions, private clinics, GPs offices, or the like, this can be extended to any environment in which either humans or animals are treated or otherwise provided with services.

Persons skilled in the art will appreciate that numerous variations and modifications will become apparent. All such variations and modifications which become apparent to persons skilled in the art, should be considered to fall within the spirit and scope that the invention broadly appearing before described.

1-94. (canceled)

95) A device for scanning medical instruments to detect a code, the device including:
a) a number of illumination sources for illuminating a surface of the instrument;
b) an emitter/detector element for:
i) generating a radiation beam to expose an area of the surface of the instrument; and,
ii) detecting reflected radiation to thereby sense the code applied to the instrument; and,
c) a shroud including a diffuser that diffuses radiation from the illumination sources, the diffuser having an opening aligned with the emitter/detector element for allowing the radiation beam to pass therethrough.

96) A scanner device according to claim 95, wherein the diffuser is a diffuser cone.

97) A scanner device according to claim 95, wherein shroud includes an outer housing.

98) A scanner device according to claim 97, wherein the outer housing helps reduce stray reflections from impinging on the emitter/detector element.

99) A scanner device according to claim 97, wherein the outer housing has a reflective inner surface for reflecting diffused radiation from the illumination sources.

100) A scanner device according to claim 99, wherein the diffused radiation is reflected to ensure that the diffused radiation is incident on the surface of the instrument at a range of angles substantially over an entire region surrounding the code.

101) A scanner device according to claim 95, wherein the shroud is for:
a) reducing stray reflections impinging on the emitter/detector element; and,
b) providing a constant intensity of background radiation reflected from the instrument surface.

102) A scanner device according to claim 95, wherein the scanner includes a body, the shroud being coupled to the body.

103) A scanner device according to claim 95, wherein the emitter/detector element generates a laser beam.

104) A scanner device according to claim 95, wherein the scanning device is for scanning barcodes.

105) A device for scanning instruments to detect a code, the device including:
a) a number of illumination sources for illuminating a surface of the instrument;
b) an emitter/detector element for:
i) generating a radiation beam to expose an area of the surface of the instrument; and,
ii) detecting reflected radiation to thereby sense the code applied to the instrument; and,
c) a shroud for:
i) reducing stray reflections impinging on the emitter/detector element; and,
ii) providing a constant intensity of background radiation reflected from the instrument surface.

106) A device according to claim 105, wherein the shroud diffuses radiation from the illumination sources and reflects the diffused radiation to ensure that the diffused radiation is incident on the surface of the instrument at a range of angles substantially over an entire region surrounding the code.

107) A shroud for a scanning device for scanning instruments to detect a code, the shroud including a diffuser that diffuses radiation from a number of illumination sources that illuminate a surface of the instrument, the diffuser having an opening aligned with an emitter/detector element to allow:
a) a radiation beam generated by the emitter/detector element to pass through the opening and expose an area of the surface of the instrument and,
b) reflected radiation to be sensed by the emitter/detector element.

108) A scanner for scanning 2-D codes, the scanner including:
a) a laser emitter/detector element that:
i) generates a laser beam which exposes an area of the surface of the instrument;
ii) detects reflected radiation and uses this to sense the 2-D code;
b) a number of illumination sources to allow the surface of the instrument to be illuminated;
c) a shroud including:
i) a diffuser cone having an opening aligned with the laser emitter/detector element to allow the laser beam to pass through the opening, and wherein the diffuser cone diffuses radiation from the number of illumination sources; and,
ii) an outer housing having a reflective inner surface for reflecting diffused radiation from the illumination sources such that the reflected diffused radiation impinges on the surface of the instrument.

109) A scanner according to claim 108, wherein the shroud diffuses and reflects the radiation from the number of radiation sources to ensure that the radiation is incident on the instrument at a range of angles substantially over an entire region surrounding the 2-D code being detected.

110) A scanner according to claim 108, wherein the shroud helps reduce the chance of stray reflections impinging on the laser emitter/detector element, and provides a constant intensity of background radiation reflected from the entire instrument surface.

111) A scanner according to claim 108, wherein the scanner includes processing for allowing an output indicative of the 2-D code or information encoded therein.

112) A method of laser marking an instrument for use in an instrument tracking system, wherein a number of instruments are provided adjacent respective apertures provided in an aperture array, and wherein the method includes, in the processing system:
a) receiving an indication of the number of apertures;
b) for each of the number of apertures, determining a unique identifier;
c) encoding each unique identifier as a respective machine readable code; and,
d) controlling a laser to thereby selectively expose bonding material provided on each instrument to thereby apply the respective machine readable code on the surface of the instrument.

113) A method according to claim 112, wherein the method includes, in the processing system, selectively positioning the laser to thereby position the focal spot aligned with the aperture.

114) A method according to claim 112, wherein the method includes, in the processing system:
a) receiving image data from an imaging system;
b) determining, using the image data, the relative position of the aperture relative to the laser; and,
c) selectively positioning the laser based on the relative position.

115) A method according to claim 112, wherein the method includes, applying the bonding material by:
a) selectively positioning a bonding material application system adjacent an aperture; and,
b) causing the bonding material application system to apply bonding material to the instrument.

116) A method according to claim 112, wherein the method includes:
a) receiving indicating data from a scanning device, the indicating data being indicative of the unique identifier and being determined when the scanning device is used to scan the code; and,
b) confirming the unique identifier matches the identifier used to generate the code, thereby confirming the machine readability of the code.

117) A method according to claim 112, wherein the method includes, in the processing system:
a) generating instrument data indicative of the instrument; and,
b) recording an association between the unique identifier and the instrument data.

118) A method according to claim 117, wherein the method further includes, in the processing system:
a) receiving indicating data from a scanning device, the indicating data being indicative of the unique identifier and being determined when the scanning device is used to scan the tag;
b) obtaining, from a database and using the indicating data, the instrument data associated with the instrument;
c) displaying at least some of the instrument data; and,
d) receiving, via an input device, input commands confirming that the instrument has been correctly identified; and,
e) making the instrument available for use following a successful confirmation.

119) A method according to claim 112, wherein the method includes determining the unique identifier by at least one of:
a) obtaining the unique identifier from another processing system; and,
b) generating the unique identifier using a predetermined algorithm.

120) Apparatus for use in laser marking an instrument for use in an instrument tracking system, wherein a number of instruments are provided adjacent respective apertures provided in an aperture array, and wherein the apparatus includes a processing system for:
a) receiving an indication of the number of apertures;
b) for each aperture, determining a unique identifier;
c) encoding each unique identifier as a respective machine readable code; and,
d) controlling a laser to thereby selectively expose bonding material provided on each instrument to thereby apply the respective machine readable code to the surface of the instrument.

121) Apparatus for use in laser marking a medical instrument having a marking area provided thereon, the apparatus including:
a) a frame having at least two apertures therein;
b) at least one support member for each aperture, each support member including
i) a shaft;
ii) a housing;
iii) a mounting pad coupled to the housing; and
iv) a resilient member for urging the mounting pad towards the frame, to thereby urge a medical instrument against the frame such that the marking area is aligned with the aperture.

122) Apparatus according to claim 121, wherein the mounting pad is pivotally mounted to the housing.

123) Apparatus according to claim 122, wherein the apparatus includes:
a) a marking system mounted to the frame and arranged so as to expose the instrument, through the aperture, using a radiation beam; and,
b) a controller for controlling the radiation beam to thereby apply the code to the instrument.

124) Apparatus according to claim 123, wherein the apparatus includes:
a) a laser for generating a laser beam; and,
b) an optical system for focussing the laser beam into a marking area aligned with the aperture.

125) Apparatus according to claim 124, wherein the laser is a CO₂ laser, and wherein the optical system is a high power density focusing optics system.

126) Apparatus according to claim 123, wherein marking system includes an imaging system, and wherein the imaging system is adapted to generate an image used in aligning the radiation beam with a selected one or the apertures.

127) Apparatus according to claim 123, wherein the code is applied to a bonding material.

128) Apparatus according to claim 127, wherein the marking system includes a bonding material application system for applying the bonding material to the instrument.

129) Apparatus according to claim 128, wherein the bonding material application system is formed from a printing system.

130) Apparatus according to claim 123, wherein the marking system includes a scanning device for:
a) scanning a code provided on an instrument; and,
b) providing indicating data indicative of the unique identifier, to a processing system.

131-141. (canceled)
